(19) United States
(12) Patent Application Publication
(10) Pub. No.: US 2023/0137065 A1
(43) Pub. Date: May 4, 2023

(54) GAS SENSOR SYSTEM, GAS SENSOR CALIBRATION METHOD, AND GAS SENSOR CALIBRATION PROGRAM

(71) Applicant: Asahi Kasei Microdevices Corporation, Tokyo (JP)

(72) Inventors: Yuta TAKAGI, Tokyo (JP); Soichi MEJIMA, Tokyo (JP)

(21) Appl. No.: 17/974,549

(22) Filed: Oct. 27, 2022

(30) Foreign Application Priority Data
Oct. 29, 2021 (JP) 2021-178213
Oct. 26, 2022 (JP) 2022-171091

(51) Int. Cl. G01N 33/00

(52) U.S. Cl. CPC G01N 33/004

(57) ABSTRACT

There is provided a gas sensor system including a first gas sensor apparatus including a transmission unit configured to transmit first calibration information and reliability information of the first gas sensor apparatus, and a second gas sensor apparatus including a reception unit configured to receive the first calibration information and the reliability information and a calibration unit configured to calibrate, based on the first calibration information, a gas concentration of a second gas sensor, in which when the calibration reliability of the first gas sensor apparatus is higher than a reference calibration reliability, the calibration unit is configured to calibrate, based on the first calibration information, the gas concentration of the second gas sensor.

The contents of the following Japanese patent application(s) are incorporated herein by reference:

NO. 2021-178213 filed in JP on Oct. 29, 2021

NO. 2022-171091 filed in JP on Oct. 26, 2022

### BACKGROUND

### 1. Technical Field

The present invention relates to a gas sensor system, a gas sensor calibration method, and a gas sensor calibration program.

### 2. Related Art

Patent document 1 describes “an accurate carbon dioxide concentration measurement system is to be provided” (summary).

### LIST OF CITED REFERENCES

### Patent Document

Patent document 1: Japanese Patent Application Publication No. 2014-228518

The summary clause does not necessarily describe all necessary features of the embodiments of the present invention. The present invention may also be a sub-combination of the features described above.

### BRIEF DESCRIPTION OF THE DRAWINGS

FIG. 1 illustrates an example of calibration of gas concentrations in a first gas sensor apparatus 100 and a second gas sensor apparatus 200 according to an embodiment of the present invention.

FIG. 2 is a block diagram illustrating an example of a gas sensor system 400 according to an embodiment of the present invention.

FIG. 3 is a block diagram illustrating another example of the gas sensor system 400 according to an embodiment of the present invention.

FIG. 4 is a block diagram illustrating another example of the gas sensor system 400 according to an embodiment of the present invention.

FIG. 5 is a block diagram illustrating another example of the gas sensor system 400 according to an embodiment of the present invention.

FIG. 6 illustrates an example of calibration of gas concentrations in the first gas sensor apparatus 100, the second gas sensor apparatus 200, and a third gas sensor apparatus 300 according to an embodiment of the present invention.

FIG. 7 illustrates an example of calibration of gas concentrations in a gas sensor apparatus 150 and a gas sensor apparatus 250 according to an embodiment of the present invention.

FIG. 8 is a block diagram illustrating an example of the gas sensor apparatus 150 and the gas sensor apparatus 250 according to an embodiment of the present invention.

FIG. 9 is a block diagram illustrating another example of the gas sensor apparatus 150 and the gas sensor apparatus 250 according to an embodiment of the present invention.

FIG. 10 is a flowchart illustrating an example of a gas sensor calibration method according to an embodiment of the present invention.

FIG. 11 illustrates an example of a detail of calibration step S104 in FIG. 10.

FIG. 12 is a flowchart illustrating another example of the gas sensor calibration method according to an embodiment of the present invention.

FIG. 13 is a flowchart illustrating an example of the gas sensor calibration method according to an embodiment of the present invention.

FIG. 14 is a flowchart illustrating an example of the gas sensor calibration method according to an embodiment of the present invention.

FIG. 15 illustrates an example of a detail of calibration step S304 in FIG. 14.

FIG. 16 is a flowchart illustrating another example of the gas sensor calibration method according to an embodiment of the present invention.

FIG. 17 illustrates an example of a computer 2200 in which the gas sensor system 400, the gas sensor apparatus 150, or the gas sensor apparatus 250 according to an embodiment of the present invention may be entirely or partially embodied.

### DESCRIPTION OF EXEMPLARY EMBODIMENTS

Hereinafter, the present invention will be described through embodiments of the invention, but the following embodiments are not intended to limit the invention according to claims. In addition, not all of the combinations of features described in the embodiments are essential to solving means of the invention.

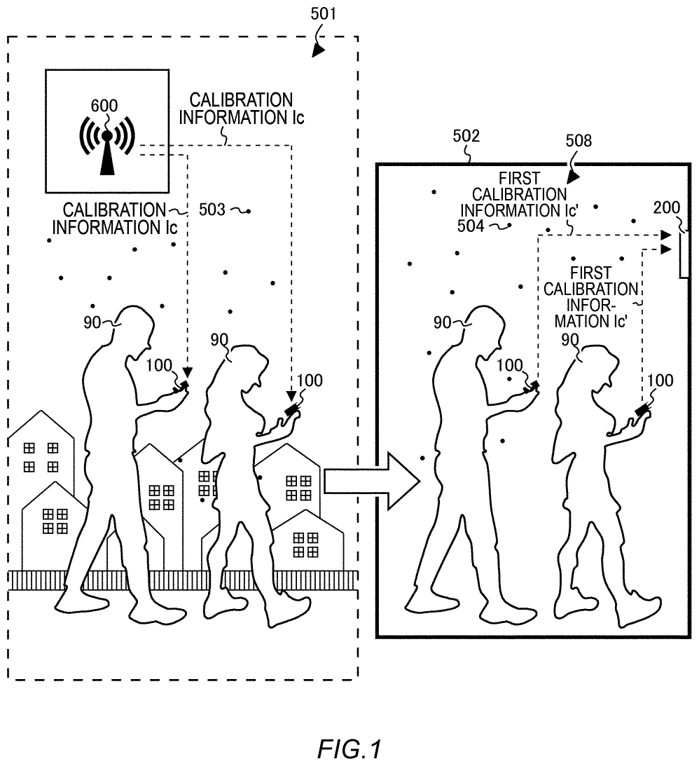

FIG. 1 illustrates an example of calibration of gas concentrations in a first gas sensor apparatus 100 and a second gas sensor apparatus 200 according to an embodiment of the present invention. In the present example, a measurement target 501 is outdoor, and a measurement target 502 is indoor.

In the present example, a living body 90 has the first gas sensor apparatus 100. The first gas sensor apparatus 100 may be a mobile terminal. The living body 90 is, for example, a person. FIG. 1 illustrates a situation where the living body 90 having the first gas sensor apparatus 100 is moving from the measurement target 501 to the measurement target 502.

A gas 503 is present in the measurement target 501. The gas 503 may be CO₂ (carbon dioxide) gas, may be CH₄ (methane) gas, or may be alcohol. A gas sensor 600 configured to measure a concentration of the gas 503 in the measurement target 501 may be arranged in the measurement target 501. When the measurement target 501 is outdoor, the gas sensor 600 measures the concentration of the gas 503 in outdoor air. The gas sensor 600 is, for example, an optical element. The gas sensor 600 may be a gas sensor based on non dispersive infrared (NDIR), may be a gas sensor based on optoacoustic spectroscopy, may be a gas sensor configured to detect a gas by a solid electrolyte, or may be a micro electro mechanical systems (MEMS) gas sensor. A detection method for the gas by the gas sensor 600 is not particularly limited.

A gas 504 is present in an interior space 508 in the measurement target 502. The gas 504 may be CO₂ (carbon dioxide) gas, may be CH₄ (methane) gas, or may be alcohol. The interior space 508 may be a space isolated from the measurement target 501. The interior space 508 may be a closed space. The interior space 508 is, for example, a room.

The second gas sensor apparatus 200 is arranged in the measurement target 502. In the present example, the second gas sensor apparatus 200 is arranged in the interior space 508. The second gas sensor apparatus 200 may be a mobile terminal.

A characteristic of the gas sensor 600 may change over a lapse of time. The characteristic of the gas sensor 600 refers to a characteristic of the optical element or the like when the gas sensor 600 is an optical element and is also a CO₂ (carbon dioxide) sensor configured to measure a gas concentration by infrared light. The characteristic of the optical element or the like may change over time. Thus, the gas sensor 600 is preferably calibrated. The gas sensor 600 is preferably regularly calibrated.

The gas sensor 600 may be subjected to self-calibration, or may be calibrated by another gas sensor 600. The self-calibration refers to calibration of the gas sensor 600 itself based on a value of the gas concentration calculated by the gas sensor 600. For the calibration of the gas sensor 600 itself, for example, a certain value of the gas concentration during a predetermined period, a maximum value or a minimum value of the gas concentration during the predetermined period, or the like may be used. For example, when the gas sensor 600 is a CO₂ (carbon dioxide) sensor, an output of the gas sensor 600 may be subjected to the self-calibration by an automatic baseline correction or automatic background calibration (ABC) algorithm.

When the measurement target 501 is outdoor and the gas sensor 600 is a CO₂ (carbon dioxide) sensor, the output of the gas sensor 600 may be calibrated at timing at which likelihood that a CO₂ (carbon dioxide) concentration in the air becomes a reference value of the CO₂ (carbon dioxide) concentration (for example, 400 ppm) is high. The output of the gas sensor 600 may be calibrated such that a CO₂ (carbon dioxide) concentration to be calculated based on the output of the gas sensor 600 indicates the reference value at the timing. The timing is, for example, a time slot in which an activity of the living body 90 is likely to be suppressed (for example, late at night).

The gas sensor 600 may transmit calibration information related to the calibration of the gas sensor 600. The calibration information is set as calibration information Ic. The calibration information Ic is calibration information for calibrating the concentration of the gas 503 in the measurement target 501 which is calculated based on the output of the gas sensor 600. The calibration information Ic is information related to calibration for causing the concentration of the gas 503 to approach a true value of the concentration. When the gas sensor 600 is a CO₂ (carbon dioxide) sensor, the calibration information Ic may be calibration information for calibrating the CO₂ (carbon dioxide) concentration such that the CO₂ (carbon dioxide) concentration to be calculated based on the output of the gas sensor 600 indicates the reference value at timing at which likelihood that a true value of the CO₂ (carbon dioxide) concentration becomes a reference value (for example, 400 ppm) is high. The gas sensor 600 may wirelessly transmit the calibration information Ic to an open space in the measurement target 501.

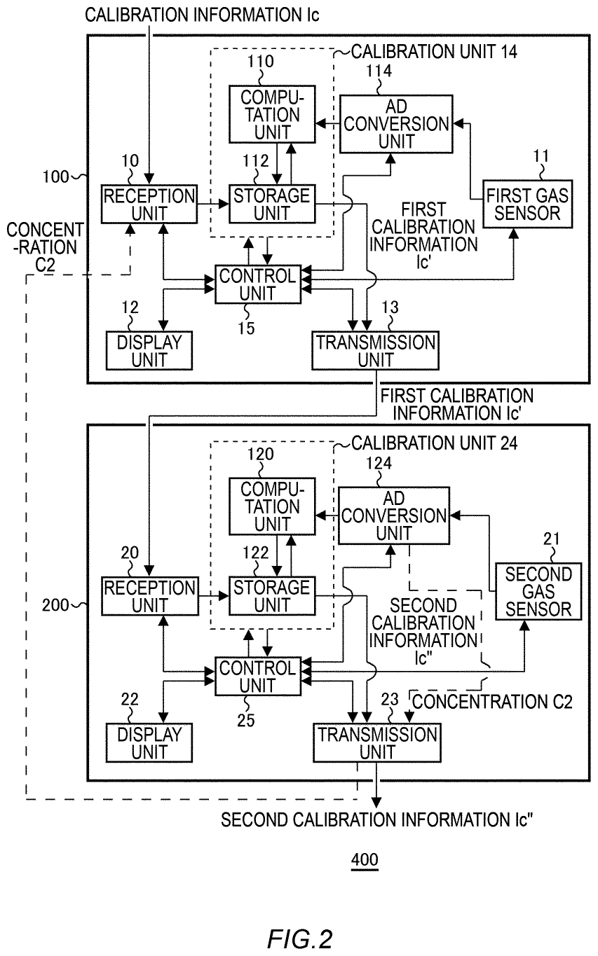

FIG. 2 is a block diagram illustrating an example of a gas sensor system 400 according to an embodiment of the present invention. The gas sensor system 400 includes the first gas sensor apparatus 100 and the second gas sensor apparatus 200. The first gas sensor apparatus 100 has a transmission unit 13. The first gas sensor apparatus 100 may have a reception unit 10, a control unit 15, a calibration unit 14, a display unit 12, and an AD conversion unit 114.

The control unit 15 is, for example, a central processing unit (CPU). The first gas sensor apparatus 100 may be a mobile terminal including the CPU. The mobile terminal may include a portable computer such as a smartphone or a tablet. The first gas sensor apparatus 100 may be a mobile terminal including the CPU, a memory, an interface, and the like.

The display unit 12 is, for example, a display, a monitor, or the like. When the first gas sensor apparatus 100 is a mobile terminal, the display unit 12 may be a display of the mobile terminal.

The first gas sensor apparatus 100 may have a first gas sensor 11. When the first gas sensor apparatus 100 is arranged in the measurement target 501, the first gas sensor 11 is configured to measure a concentration of the gas 503 in the measurement target 501. The first gas sensor 11 may be a CO₂ (carbon dioxide) sensor, may be a CH₄ (methane) sensor, or may be an alcohol sensor. The first gas sensor 11 may measure the gas 503 (see FIG. 1) of a same type as that for the gas sensor 600 (see FIG. 1). That is, when the gas sensor 600 is a CO₂ (carbon dioxide) sensor, the first gas sensor 11 may be a CO₂ (carbon dioxide) sensor.

A characteristic of the first gas sensor 11 may change over a lapse of time. Thus, the first gas sensor 11 is preferably calibrated. In the present example, the reception unit 10 is configured to receive the calibration information Ic transmitted by the gas sensor 600. In the present example, the calibration unit 14 is configured to calibrate, based on the calibration information Ic, the concentration of the gas 503 (see FIG. 1) in the measurement target 501 (see FIG. 1) which is calculated based on an output of the first gas sensor 11. As a result, the measurement value of the concentration of the gas 503 by the first gas sensor 11 is more likely to be an accurate value than before the calibration. The calibration unit 14 may calibrate the concentration of the gas 503 (see FIG. 1) in a state where the first gas sensor apparatus 100 is arranged in the measurement target 501 (in the present example, a state where the living body 90 is present outdoor).

The AD conversion unit 114 is configured to convert an output of an analog signal of the first gas sensor 11 into a digital signal. The calibration unit 14 may calibrate, based on the calibration information Ic, the concentration of the gas 503 (see FIG. 1) which is calculated based on the output of the first gas sensor 11. That is, after the concentration of the gas 503 is calculated, the calibration unit 14 may calibrate the calculated concentration of the gas 503. While calculating the concentration of the gas 503, the calibration unit 14 may calibrate the concentration of the gas 503 based on the output of the first gas sensor 11 and the calibration information Ic.

In the present example, the calibration unit 14 includes a computation unit 110 and a storage unit 112. The computation unit 110 is configured to calculate the concentration of the gas 503 based on the digital signal of the first gas sensor 11 which is converted by the AD conversion unit 114. The storage unit 112 is configured to store the concentration of the gas 503 which is calculated by the computation unit 110. The storage unit 112 may store a correlation between the concentration of the gas 503 which is calculated by the computation unit 110 and the calibration information Ic. The correlation is set as a correlation Cr. The correlation Cr may be a correlation function or may be a correlation table.

The computation unit 110 may compute first calibration information Ic′ based on the calibration information Ic received by the reception unit 10 and the correlation Cr stored in the storage unit 112. The first calibration information Ic′ is calibration information for calibrating the concentration of the gas 503 (see FIG. 1) of the measurement target 501 (see FIG. 1) which is calculated based on the output of the first gas sensor 11. The first calibration information Ic′ is information related to calibration for causing the concentration of the gas 503 (see FIG. 1) to approach a true value of the concentration. The calibration information Ic′ may be different from or may be identical to the calibration information Ic.

The computation unit 110 is, for example, a central processing unit (CPU). The computation unit 110 and the control unit 15 may be a single CPU.

The transmission unit 13 is configured to transmit the first calibration information Ic′ of the first gas sensor 11. When the living body 90 (see FIG. 1) remains in the measurement target 501 (see FIG. 1), the transmission unit 13 may transmit the first calibration information Ic′ to the open space in the measurement target 501. When the living body 90 has moved from the measurement target 501 to the measurement target 502 (see FIG. 1), the transmission unit 13 may transmit the first calibration information Ic′ to the interior space 508. The transmission unit 13 may wirelessly transmit the first calibration information Ic′.

Note that when the first gas sensor apparatus 100 does not have the first gas sensor 11, a configuration may be adopted where the first gas sensor apparatus 100 does not have the AD conversion unit 114 too. When the first gas sensor apparatus 100 does not have the first gas sensor 11 and the AD conversion unit 114, the output of the analog signal of the first gas sensor 11 may be converted into a digital signal by the AD conversion unit 114 arranged outside the first gas sensor apparatus 100. The digital signal converted by the AD conversion unit 114 may be transmitted to the first gas sensor apparatus 100. The same also applies when the second gas sensor apparatus 200 does not have a second gas sensor 21 (described below).

The second gas sensor apparatus 200 has a reception unit 20 and a calibration unit 24. The second gas sensor apparatus 200 may have a control unit 25, a display unit 22, a transmission unit 23, and an AD conversion unit 124. Functions of the control unit 25, the display unit 22, and the transmission unit 23 may be respectively the same as those of the control unit 15, the display unit 12, and the transmission unit 13 in the first gas sensor apparatus 100.

The reception unit 20 is configured to receive the first calibration information Ic′ transmitted by the transmission unit 13 of the first gas sensor apparatus 100. The second gas sensor apparatus 200 may have the second gas sensor 21. The calibration unit 24 is configured to calibrate, based on the first calibration information Ic′ received by the reception unit 20, a concentration of the gas 504 (see FIG. 1) of the measurement target 502 (see FIG. 1) which is calculated based on an output of the second gas sensor 21. As a result, a measurement value of the concentration of the gas 504 by the second gas sensor 21 is more likely to be an accurate value than before the calibration.

The AD conversion unit 124 is configured to convert an output of an analog signal of a second gas sensor 21 into a digital signal. The calibration unit 24 may calibrate, based on the first calibration information Ic′, the concentration of the gas 504 (see FIG. 1) which is calculated based on the output of the second gas sensor 21. That is, after the concentration of the gas 504 is calculated, the calibration unit 24 may calibrate the calculated concentration of the gas 504. While calculating the concentration of the gas 504, the calibration unit 24 may calibrate the concentration of the gas 504 based on the output of the second gas sensor 21 and the first calibration information Ic′.

As described above, the first calibration information Ic′ is the calibration information for calibrating the concentration of the gas 503 (see FIG. 1) in the measurement target 501 (see FIG. 1) which is calculated based on the output of the first gas sensor 11. When the calibration unit 14 calibrates the concentration of the gas 503 (see FIG. 1) in a state where the first gas sensor apparatus 100 is arranged in the measurement target 501 (in the present example, a state where the living body 90 is present outdoor) and also the first gas sensor apparatus 100 has moved from the measurement target 501 to the measurement target 502, likelihood that the first gas sensor 11 can accurately measure the concentration of the gas 504 in the measurement target 502 is high. Thus, based on the first calibration information Ic′, the calibration unit 24 of the second gas sensor apparatus 200 is likely to accurately calibrate the concentration of the gas 504 which is calculated based on the output of the second gas sensor 21.

The gas sensor system 400 may include a storage unit 122. In the present example, the second gas sensor apparatus 200 includes the storage unit 122. In the present example, the calibration unit 24 includes a computation unit 120 and the storage unit 122. Functions of the computation unit 120 and the storage unit 122 may be respectively the same as those of the computation unit 110 and the storage unit 112 in the calibration unit 14 of the first gas sensor apparatus 100.

The storage unit 122 may store a correlation between the concentration of the gas 504 which is calculated by the computation unit 120 and the first calibration information Ic′. The correlation is set as a correlation Cr′. The computation unit 120 may compute second calibration information Ic″ based on the first calibration information Ic′ received by the reception unit 20 and the correlation Cr′ stored in the storage unit 122.

The second calibration information Ic″ is calibration information for calibrating the concentration of the gas 504 (see FIG. 1) in the measurement target 502 (see FIG. 1) which is calculated based on the output of the second gas sensor 21. The second calibration information Ic″ is information related to calibration for causing the concentration of the gas 504 (see FIG. 1) to approach a true value of the concentration. The second calibration information Ic″ may be different from or may be identical to the first calibration information Ic′.

In the gas sensor system 400, the reception unit 20 is configured to receive the first calibration information Ic′, and also the calibration unit 24 is configured to calibrate, based on the first calibration information Ic′, the concentration of the gas 504 (see FIG. 1) which is calculated based on the output of the second gas sensor 21. As a result, the gas sensor system 400 can calibrate the concentration of the gas 504 which is calculated based on the output of the second gas sensor 21.

When the interior space 508 (see FIG. 1) is a closed space isolated from outdoor, a case is conceivable that the CO₂ (carbon dioxide) concentration in the interior space 508 does not become the above described reference value (for example, 400 ppm) even in a time slot in which the activity of the living body 90 is likely to be suppressed (for example, late at night). When the CO₂ (carbon dioxide) concentration in the interior space 508 does not become the reference value, it is difficult for the second gas sensor 21 to perform the self-calibration of the measured concentration of the gas 504 (see FIG. 1).

In the present example, in a state where the first gas sensor apparatus 100 is arranged in the measurement target 501 (in the present example, a state where the living body 90 is present outdoor), the concentration of the gas 503 (see FIG. 1) which is calculated based on the output of the first gas sensor 11 is calibrated by the calibration unit 14. Thus, when the living body 90 has moved from the measurement target 501 (see FIG. 1) to the interior space 508 (see FIG. 1), the first gas sensor apparatus 100 is likely to accurately measure the concentration of the gas 504 in the interior space 508.

In the present example, after the living body 90 has moved to the interior space 508, the transmission unit 13 transmits the first calibration information Ic′. Thus, even when the second gas sensor apparatus 200 is arranged in an environment (in the present example, the interior space 508) where it is difficult to perform the self-calibration, the gas sensor system 400 can calibrate the concentration of the gas 504 which is calculated based on the output of the second gas sensor 21. The gas sensor system 400 can calibrate the concentration of the gas 504 which is calculated based on the output of the second gas sensor 21 in a state where the second gas sensor apparatus 200 is arranged in the measurement target 502 (see FIG. 1).

The calibration information Ic may be a reading of the concentration of the gas 503 (see FIG. 1) in the measurement target 501 (see FIG. 1). The reading of the concentration of the gas 503 may be a concentration of the gas 503 which is calculated based on the output of the gas sensor 600 (see FIG. 1).

A predetermined distance between the gas sensor 600 and the first gas sensor 11 is set as a distance dp1. When the distance between the gas sensor 600 and the first gas sensor 11 is less than the distance dp1, likelihood that the gas sensor 600 and the first gas sensor 11 are arranged in the measurement target 501 with the same concentration of the gas 503 is high. Thus, the concentration of the gas 503 which is calculated based on the output of the gas sensor 600 and the concentration of the gas 503 which is calculated based on the output of the first gas sensor 11 are preferably identical. Thus, when the distance between the gas sensor 600 and the first gas sensor 11 is less than the distance dp1, the calibration information Ic may be a reading of the concentration of the gas 503 in the measurement target 501. The distance dp1 is, for example, 10 m. When the gas sensor 600 and the first gas sensor 11 are arranged in the interior space 508, the distance dp1 may be 5 m.

The calibration information Ic may be a calibration amount of the concentration of the gas 503 (see FIG. 1) in the measurement target 501 (see FIG. 1). The concentration of the gas 503 which is calculated based on the output of the gas sensor 600 (see FIG. 1) is set as a concentration C0. The concentration of the gas 503 which is calculated based on the output of the first gas sensor 11 is set as a concentration C1. The calibration amount of the concentration of the gas 503 may be a difference between the concentration C0 and the concentration C1.

When the calibration amount of the concentration of the gas 503 is a difference between the concentration C0 and the concentration C1, the calibration unit 14 may calibrate, by the difference, the output of the gas 503 by the first gas sensor 11. The calibration unit 14 may offset the output of the gas 503 by the first gas sensor 11 by the difference.

When it is assumed that a state of the gas sensor 600 is comparable to a state of the first gas sensor 11, the calibration information Ic is preferably a calibration amount of the concentration of the gas 503 (see FIG. 1). A situation where the state of the gas sensor 600 is comparable to the state of the first gas sensor 11 is, for example, a case where likelihood that a deterioration state of the gas sensor 600 is comparable to a deterioration state of the first gas sensor 11 is high. A case where the likelihood that the deterioration state of the gas sensor 600 is comparable to the deterioration state of the first gas sensor 11 is high may be a case where at least one of a case where a most recent calibration period of the gas sensor 600 is the same as a most recent calibration period of the first gas sensor 11, a case where an environment in which the gas sensor 600 is arranged is similar to an environment in which the first gas sensor 11 is arranged, or a case where a specification of the gas sensor 600 is the same as a specification of the first gas sensor 11 is satisfied. The environment in which the gas sensor 600 is arranged may refer to at least one of a temperature, a humidity, or a concentration of the gas 503 in a location where the gas sensor 600 is arranged.

The first calibration information Ic′ may be a reading of the concentration of the gas 504 (see FIG. 1) in the measurement target 502 (see FIG. 1). The reading of the concentration of the gas 504 may be a concentration of the gas 504 which is calculated based on the output of the first gas sensor 11.

A predetermined distance between the first gas sensor 11 and the second gas sensor 21 is set as a distance dp2. Similarly as in the case of the calibration information Ic, when the distance between the first gas sensor 11 and the second gas sensor 21 is less than the distance dp2, the first calibration information Ic′ may be a reading of the concentration of the gas 504 in the measurement target 502.

Similarly as in the case of the calibration information Ic, the first calibration information Ic′ may be a calibration amount of the concentration of the gas 504 (see FIG. 1) in the measurement target 502 (see FIG. 1). When it is assumed that the state of the first gas sensor 11 is comparable to the state of the second gas sensor 21, the first calibration information Ic′ is preferably the calibration amount of the concentration of the gas 504 due to a reason similar to the above described reason with regard to the case of the calibration information Ic.

A concentration of the gas 504 (see FIG. 1) which is calculate based on the output of the first gas sensor 11 is set as a concentration C1′. A concentration of the gas 504 which is calculated based on the output of the second gas sensor 21 is set as a concentration C2. When the calibration amount of the concentration of the gas 504 is a difference between the concentration C1′ and the concentration C2, the calibration unit 24 may calibrate, by the difference, the concentration C2 calculated based on the output of the second gas sensor 21.

The transmission unit 23 of the second gas sensor apparatus 200 may transmit the concentration C2 to the first gas sensor apparatus 100. In FIG. 2, a path in which the concentration C2 is to be transmitted to the first gas sensor apparatus 100 is represented by a coarse dotted arrow. When the transmission unit 23 transmits the concentration C2 to the first gas sensor apparatus 100, the concentration C2 may be a concentration of the gas 504 before being calibrated by the calibration unit 24. The reception unit 10 of the first gas sensor apparatus 100 may receive the concentration C2. The computation unit 110 of the first gas sensor apparatus 100 may compute a difference between the concentration C1′ and the concentration C2. The transmission unit 13 may transmit the difference to the reception unit 20. The calibration unit 24 may calibrate, by the difference, the concentration C2 received by the reception unit 20.

The first gas sensor apparatus 100 may be subjected to self-calibration. When the first gas sensor 11 is a CO₂ (carbon dioxide) sensor, the first gas sensor apparatus 100 may be subjected to the self-calibration by an automatic baseline correction or automatic background calibration (ABC) algorithm. When the measurement target 501 (see FIG. 1) is an environment in which the self-calibration can be performed (for example, outdoor) and the first gas sensor apparatus 100 is arranged in the measurement target 501, the first gas sensor apparatus 100 may be subjected to the self-calibration in a state of being arranged in the measurement target 501. The concentration C2 to be calculated based on the output of the second gas sensor 21 may be calibrated based on the first calibration information Ic′ of the first gas sensor 11 on which the self-calibration has been performed.

The first gas sensor apparatus 100 may or may not include the first gas sensor 11. In the present example, the first gas sensor apparatus 100 includes the first gas sensor 11. When the first gas sensor apparatus 100 does not include the first gas sensor 11, the output of the first gas sensor 11 may be transmitted to the calibration unit 14. The same also applies to the second gas sensor apparatus 200.

In the present example, the measurement target 502 in which the concentration C1′ is calculated based on the output of the first gas sensor 11 is the same as the measurement target 502 in which the concentration C2 is calculated based on the output of the second gas sensor 21. A situation where the measurement target 502 in which the concentration C1′ is calculated is the same as the measurement target 502 in which the concentration C2 is calculated may refer to a situation where a type of a measurement target gas of the first gas sensor apparatus 100 is the same as a type of a measurement target gas of the second gas sensor apparatus 200 in the measurement target 502. The measurement target gas of the first gas sensor apparatus 100 and the second gas sensor apparatus 200 may be CO₂ (carbon dioxide), may be CH₄ (methane), or may be alcohol.

A situation where the measurement target 502 in which the concentration C1′ is calculated is the same as the measurement target 502 in which the concentration C2 is calculated may refer to a situation where the first gas sensor 11 and the second gas sensor 21 share a same space (in the present example, the interior space 508 (see FIG. 1)). The same space may refer to at least one of a case where a temperature or a humidity in the space is the same, a case where an ID of short distance radio (Wi-Fi (registered trademark) or the like) is the same, or a case where, when the first gas sensor apparatus 100 and the second gas sensor apparatus 200 are a mobile terminal, an amplitude and a frequency of acoustic waves acquired by the mobile terminal are the same.

Note that in the measurement target 502, a type of the measurement target gas the concentration C1′ of which is calculated based on the output of the first gas sensor 11 may be different from a type of the measurement target gas the concentration C2 of which is calculated based on the output of the second gas sensor 21. The type of the measurement target gas the concentration C1′ of which is calculated based on the output of the first gas sensor 11 is set as a gas type G1. The type of the measurement target gas the concentration C2 of which is calculated based on the output of the second gas sensor 21 is set as a gas type G2. When the gas type G1 is different from the gas type G2, the first calibration information Ic′ may include information of the gas type G1. The calibration unit 24 may calibrate, based on the first calibration information Ic′, the concentration of the gas of the gas type G2 which is calculated based on the output of the second gas sensor.

Note that when the first gas sensor 11 is arranged in the measurement target 501 and the second gas sensor 21 is arranged in the measurement target 502, likelihood that the concentration of the gas 503 in the measurement target 501 is different from the concentration of the gas 504 in the measurement target 502 is high. Thus, when the first gas sensor 11 is arranged in the measurement target 501 and the second gas sensor 21 is arranged in the measurement target 502, a configuration may be adopted where the calibration unit 24 does not calibrate the concentration C2 which is calculated based on the output of the second gas sensor 21.

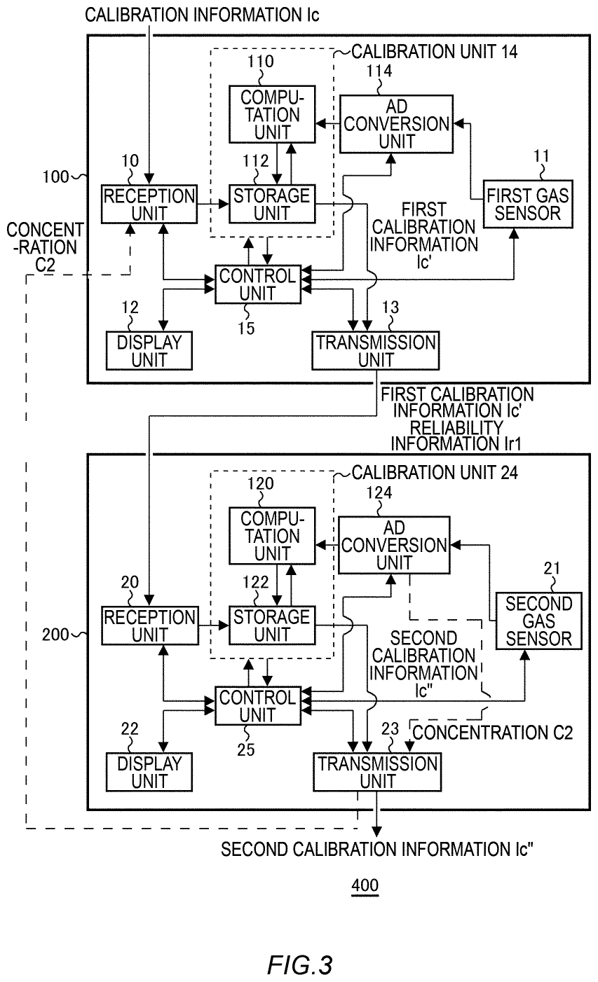

FIG. 3 is a block diagram illustrating another example of the gas sensor system 400 according to an embodiment of the present invention. A calibration reliability of the first gas sensor apparatus 100 is set as a calibration reliability R1. Reliability information indicating the calibration reliability R1 is set as reliability information Ir1. The reliability information Ir1 is information indicating, when the first gas sensor apparatus 100 is calibrated, a reliability of the calibration performed on the first gas sensor apparatus 100. The reliability information Ir1 may include at least one of information on an elapsed period of time from the most recent calibration, information on an elapsed period of time since the first gas sensor 11 is installed, information on calibration means, information related to a diversity of a calibration source, information on the number of times to perform calibration, information on a calibration frequency, information of a gas concentration at the time of calibration, or environment information at the time of calibration.

The calibration reliability R1 is more likely to be higher as the elapsed period of time from the most recent calibration is shorter. The reliability information Ir1 may be stored in the storage unit 112.

The information of the calibration means is information indicating whether the first gas sensor apparatus 100 is subjected to the self-calibration or is calibrated by another sensor (for example, the gas sensor 600 (see FIG. 1)). When the first gas sensor apparatus 100 is calibrated by another sensor, the information of the calibration means may include at least one of a distance between the first gas sensor and the other sensor or a calibration state of the other sensor. Note that in the first gas sensor apparatus 100, by calibrating the output of the first gas sensor 11 by the calibration unit 14, the self-calibration on the first gas sensor 11 may be performed.

The information related to the diversity of the calibration source may include at least one of: a number of at least one sensor (for example, the gas sensor 600 (see FIG. 1)) as the calibration source of the first gas sensor 11; or a number of at least one specification of the sensor as the calibration source. The specification of the sensor is, for example, a specific configuration of the sensor. When the first gas sensor apparatus 100 is subjected to the self-calibration, the number of sensors of the calibration source and the number of specifications of the sensor of the calibration source may include the first gas sensor 11. As the number of sensors of the calibration source is higher, the calibration reliability R1 is more likely to be increased. As the number of specifications of the sensor of the calibration source is higher, the calibration reliability R1 is more likely to be increased.

The information of the number of times to perform calibration is information related to the number of times the first gas sensor apparatus 100 has been calibrated from a predetermined time point in the past to the present. The number of times to perform calibration may include a case where the first gas sensor apparatus 100 has been subjected to the self-calibration and a case where the first gas sensor apparatus 100 has been calibrated by another sensor. As the number of times to perform calibration is higher, the calibration reliability R1 is more likely to be increased.

The information of the calibration frequency is information of the number of times to perform calibration on the first gas sensor apparatus 100 per predetermined period of time. The number of times to perform calibration may include a case where the first gas sensor apparatus 100 has been subjected to the self-calibration and a case where the first gas sensor apparatus 100 has been calibrated by another sensor. As the calibration frequency is higher, the calibration reliability R1 is more likely to be increased.

Information of the gas concentration at the time of calibration is information of a gas concentration at the time of calibration of the first gas sensor apparatus 100. The time of calibration of the first gas sensor apparatus 100 may include the time of the self-calibration of the first gas sensor apparatus 100 and the time of the calibration of the first gas sensor apparatus 100 by another sensor. When the first gas sensor apparatus 100 is calibrated at the time of measurement of a gas concentration that is not within a predetermined range (for example, an abnormal value of the gas concentration), the calibration reliability R1 is more likely to be decreased as compared with a case the calibration is performed at the time of measurement of a gas concentration at a reference value.

The environment information at the time of calibration may include a temperature, a humidity, or an air pressure of a space (for example, the measurement target 501 (see FIG. 1)) in which the first gas sensor 11 is arranged at the time of calibration of the first gas sensor apparatus 100. When the first gas sensor 11 is calibrated at a temperature that is not in a predetermined range (for example, an abnormal value of the temperature), a humidity that is not in a predetermined range (for example, an abnormal value of the humidity), or an air pressure that is not in a predetermined range (for example, an abnormal value of the air pressure), the calibration reliability R1 is more likely to be decreased as compared with a case where the calibration is performed at a temperature at a reference value, a humidity at a reference value, or an air pressure at a reference value.

The transmission unit 13 may further transmit the reliability information Ir1. The reception unit 20 of the second gas sensor apparatus 200 may further receive the reliability information Ir1. The calibration unit 24 may calibrate, based on the first calibration information Ic′ and the calibration reliability R1 of the first gas sensor apparatus 100, the concentration of the gas 504 (see FIG. 1) which is calculated based on the output of the second gas sensor 21. The calibration unit 24 may calibrate, based on the first calibration information Ic′, the concentration of the gas 504 (see FIG. 1) which is calculated based on the output of the second gas sensor 21 according to the reliability information Ir1.

A calibration reliability of the second gas sensor apparatus 200 is set as a calibration reliability R2. Reliability information indicating the calibration reliability R2 is set as reliability information Ir2. The reliability information Ir2 is information indicating, when the second gas sensor apparatus 200 is calibrated, a reliability of the calibration performed on the second gas sensor apparatus 200. The reliability information Ir2 may include information similar to the reliability information Ir1 described above. The reliability information Ir2 and the reliability information Ir1 which has been received by the reception unit 20 may be stored in the storage unit 122.

A predetermined threshold of the calibration reliability R1 is set as a first threshold Rth1. The first threshold Rth1 may be different for each of the measurement targets 501 (see FIG. 1). When the calibration reliability R1 exceeds the first threshold Rth1, a user of the gas sensor system 400 may trust the concentration of the gas 503 (see FIG. 1) which is calculated based on the output of the first gas sensor 11.

A predetermined threshold of the calibration reliability R2 is set as a second threshold Rth2. The second threshold Rth2 may be different for each of the measurement targets 502 (see FIG. 1). When the calibration reliability R2 exceeds the second threshold Rth2, the user of the gas sensor system 400 may trust the concentration of the gas 504 (see FIG. 1) which is calculated based on the output of the second gas sensor 21. Note that the second threshold Rth2 and the first threshold Rth1 may be the same or may be different from each other.

The reliability information Ir2 may include information of a reliability of self-calibration in the second gas sensor apparatus 200. The self-calibration refers to calibration that is not based on another gas sensor (in the present example, for example, the first gas sensor apparatus 100). When the second gas sensor 21 is a CO₂ (carbon dioxide) sensor, the self-calibration may refer to calibration based on an automatic baseline correction or automatic background calibration (ABC) algorithm.

The information of the reliability of the self-calibration in the second gas sensor apparatus 200 may include information on whether the second gas sensor apparatus 200 has a function of the self-calibration. The second gas sensor apparatus 200 may perform the self-calibration of the second gas sensor apparatus 200 by calibrating the output of the second gas sensor 21 by the calibration unit 24.

When the calibration reliability R1 is equal to or lower than the first threshold Rth1, a configuration may be adopted where the calibration unit 24 does not calibrate, based on the first calibration information Ic′, the concentration of the gas 504 (see FIG. 1) which is calculated based on the output of the second gas sensor 21. When the calibration reliability R2 of the self-calibration of the second gas sensor apparatus 200 is equal to or lower than the second threshold Rth2 and also the calibration reliability R1 exceeds the first threshold Rth1, the calibration unit 24 may calibrate, based on the first calibration information Ic′, the concentration of the gas 504 (see FIG. 1) which is calculated based on the output of the second gas sensor 21. A case where the calibration reliability R2 of the self-calibration of the second gas sensor apparatus 200 is equal to or lower than the second threshold Rth2 may include a case where the second gas sensor apparatus 200 does not have a function of the self-calibration. A case where the calibration reliability R2 of the self-calibration of the second gas sensor apparatus 200 is equal to or lower than the second threshold Rth2 may include a case where the self-calibration is difficult due to a reason that the second gas sensor apparatus 200 is an old sensor device, or the like.

The calibration unit 24 may compare the calibration reliability R1 of the first gas sensor apparatus 100 which his received by the reception unit 20 with the calibration reliability R2 of the second gas sensor apparatus 200. The calibration reliability R2 may include the calibration reliability R2 of the self-calibration of the second gas sensor apparatus 200 and the calibration reliability R2 in a case where the second gas sensor apparatus 200 is calibrated by another sensor.

When the calibration reliability R1 of the first gas sensor apparatus 100 is higher than the calibration reliability R2 of the second gas sensor apparatus 200, the calibration unit 24 may calibrate, based on the first calibration information Ic′ received by the reception unit 20, the concentration of the gas 504 (see FIG. 1) which is calculated based on the output of the second gas sensor 21. Since the concentration of the gas 504 is calibrated, the concentration of the gas 504 which is calculated based on the output of the second gas sensor 21 is likely to be accurate. When the calibration reliability R1 is equal to or lower than the calibration reliability R2, a configuration may be adopted where the calibration unit 24 does not calibrate the concentration of the gas 504 (see FIG. 1) which is calculated based on the output of the second gas sensor 21 but may perform calibration based on the second calibration information Ic″.

When the calibration reliability R2 is equal to or lower than the second threshold Rth2, the transmission unit 23 of the second gas sensor apparatus 200 may transmit information to another gas sensor apparatus (for example, at least one of the gas sensor 600 or the first gas sensor apparatus 100) for requesting transmission of calibration information for calibrating the output of the second gas sensor 21 to the second gas sensor apparatus 200. When the calibration reliability R2 is equal to or lower than the second threshold Rth2, the second gas sensor apparatus 200 may be in a standby state. As a result, power consumption of the second gas sensor apparatus 200 is likely to be reduced.

The calibration reliability R1 of the first gas sensor apparatus 100 which is received by the reception unit 20 may be stored in the storage unit 122. The calibration reliability R1 stored in the storage unit 122 may refer to the past calibration reliability R1 the first gas sensor apparatus 100. The calibration unit 24 may compare the calibration reliability R1 stored in the storage unit 122 with the current calibration reliability R1 of the first gas sensor apparatus 100 which is received by the reception unit 20. When the current calibration reliability R1 of the first gas sensor apparatus 100 is higher than the calibration reliability R1 of the first gas sensor apparatus 100 which is stored in the storage unit 122, the calibration unit 24 may calibrate, based on the first calibration information Ic′, the concentration of the gas 504 (see FIG. 1) which is calculated based on the output of the second gas sensor 21.

A calibration reliability serving as a reference for calibrating the concentration of the gas 504 which is calculated based on the output of the second gas sensor 21 is set as a reference calibration reliability Cs. Reference reliability information indicating the reference calibration reliability Cs is set as reference reliability information Irs. The reference calibration reliability Cs may be at least one of the calibration reliability R1 of the first gas sensor apparatus 100 or the calibration reliability R2 of the second gas sensor apparatus 200. When the calibration reliability R1 of the first gas sensor apparatus 100 which is received by the reception unit 20 is higher than the reference calibration reliability Cs, the calibration unit 24 may calibrate, based on the first calibration information Ic′, the gas concentration in the measurement target 502 which is calculated based on the output of the second gas sensor 21.

The storage unit 122 may store the reference reliability information Cs. When the calibration reliability R1 of the first gas sensor apparatus 100 which is received by the reception unit 20 is higher than the reference calibration reliability Cs, the storage unit 122 may update the reference calibration reliability Cs based on the received calibration reliability R1 and store the updated reference calibration reliability Cs. The calibration unit 24 may calibrate, based on the first calibration information Ic′, the gas concentration in the measurement target 502 which is calculated based on the output of the second gas sensor 21 according to the updated reference reliability Cs.

When the calibration reliability R1 of the first gas sensor apparatus 100 which is received by the reception unit 20 is higher than the reference calibration reliability Cs, the storage unit 122 may update the reference calibration reliability Cs to the calibration reliability R1 and store the updated reference calibration reliability Cs. When the calibration reliability R1 is lower than the reference calibration reliability Cs, a configuration may be adopted where the storage unit 122 does not update the reference calibration reliability Cs.

The reference reliability information Cs stored in the storage unit 122 may be the reference reliability information Cs updated based on the calibration reliability R1 or may be the calibration reliability R2. The calibration reliability R2 may include the calibration reliability R2 of the self-calibration of the second gas sensor apparatus 200 and the calibration reliability R2 in a case where the second gas sensor apparatus 200 is calibrated by another sensor.

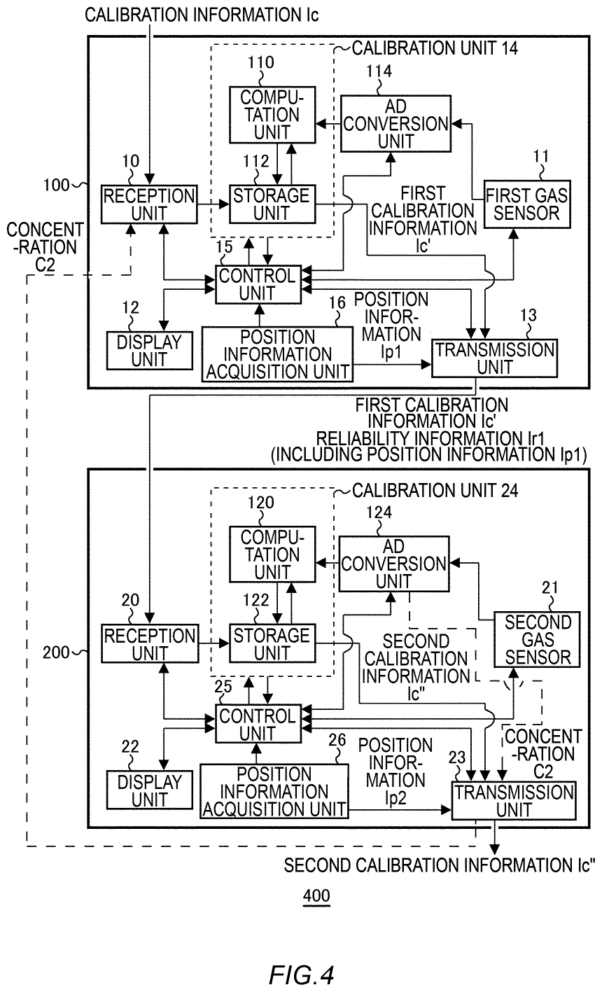

FIG. 4 is a block diagram illustrating another example of the gas sensor system 400 according to an embodiment of the present invention. In the gas sensor system 400 of the present example, the first gas sensor apparatus 100 further has a position information acquisition unit 16, and the second gas sensor apparatus 200 further has a position information acquisition unit 26. The gas sensor system 400 of the present example is different from the example illustrated in FIG. 3 in the above described aspect.

The position information acquisition unit 16 is configured to acquire information of a position of the first gas sensor apparatus 100. The position information acquisition unit 26 is configured to acquire information of a position of the second gas sensor apparatus 200. The position information acquisition unit 16 and the position information acquisition unit 26 are, for example, a global positioning system (GPS).

The position information of the first gas sensor apparatus 100 is set as position information Ip1. The reliability information Ir1 may include the position information Ip1. The position information of the second gas sensor apparatus 200 is set as position information Ip2. The reliability information Ir2 may include the position information Ip2.

A distance between the position of the first gas sensor apparatus 100 and the position of the second gas sensor apparatus 200 which is acquired by the position information acquisition unit 26 is set as a distance d. As described above, the predetermined distance between the position of the first gas sensor 11 and the position of the second gas sensor 21 is the distance dp2. The distance dp2 may refer to a distance at which likelihood that the first gas sensor 11 and the second gas sensor 21 are arranged in a same space (for example, the interior space 508 of FIG. 1) is high. The same space may refer to a situation where a type of the measurement target gas the concentration C1′ of which is calculated based on the output of the first gas sensor 11 is the same as a type of the measurement target gas the concentration C2 of which is calculated based on the output of the second gas sensor 21, or may refer to a situation where an ID of short distance radio (Wi-Fi (registered trademark) or the like) in the space where the first gas sensor 11 is arranged is the same as an ID of short distance radio (Wi-Fi (registered trademark) or the like) in the space where the second gas sensor 21 is arranged.

The calibration unit 24 may calibrate, based on the distance d and the first calibration information Ic′, the concentration of the gas 504 (see FIG. 1) which is calculated based on the output of the second gas sensor 21. When the distance d is less than the distance dp2, the calibration unit 24 may calibrate the concentration of the gas 504 which is calculated based on the output of the second gas sensor 21. When the distance d is equal to or more than the distance dp2, a configuration may be adopted where the calibration unit 24 does not calibrate the concentration of the gas 504 which is calculated based on the output of the second gas sensor 21. The calibration unit 24 may calibrate, based on the first calibration information Ic′, the concentration of the gas 504 (see FIG. 1) which is calculated based on the output of the second gas sensor 21 according to the distance d.

Note that the reliability information Ir1 may include information of a user of the first gas sensor apparatus 100. The information of the user may include a plurality of usernames and a past use history of the first gas sensor apparatus 100 for each of the usernames. The past use history may include a past use situation. The past use situation is, for example, a situation where a particular user intentionally blows exhalation to the first gas sensor apparatus 100, or the like. The calibration reliability R1 in a case where the first gas sensor apparatus 100 is used by the particular user may be lower than the calibration reliability R1 in a case where the first gas sensor apparatus 100 is used by another user. The particular user may be stored as a person on watch list in the storage unit 122.

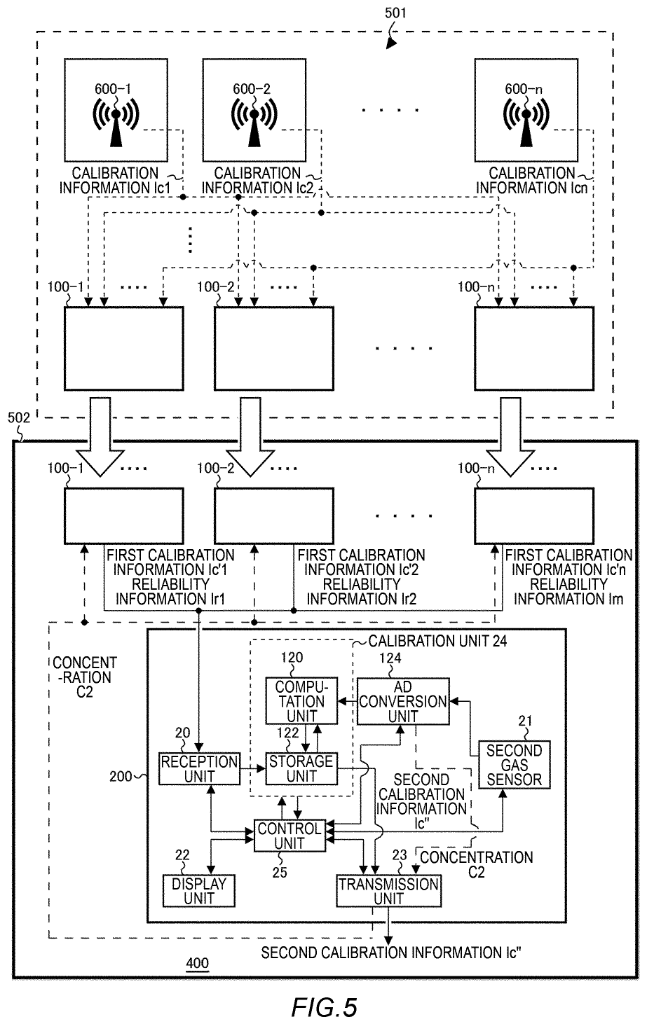

FIG. 5 is a block diagram illustrating another example of the gas sensor system 400 according to an embodiment of the present invention. The gas sensor system 400 of the present example is different from the gas sensor system 400 illustrated in FIG. 2 in that a plurality of first gas sensor apparatuses 100 (a first gas sensor apparatus 100-1 to a first gas sensor apparatus 100-n) are included. In FIG. 5, an illustration of the gas 503 in the measurement target 501 and the gas 504 in the measurement target 502 is omitted.

Each of the first gas sensor apparatus 100-1 to the first gas sensor apparatus 100-n may be possessed by each of a plurality of living bodies 90 (a living body 90-1 to a living body 90-n). The plurality of first gas sensor apparatuses 100 may be moved from the measurement target 501 to the measurement target 502 when the plurality of living bodies 90 move from the measurement target 501 to the measurement target 502.

In the present example, the first gas sensor apparatus 100-1 to the first gas sensor apparatus 100-n respectively have a reception unit 10-1 to a reception unit 10-n, respectively have a calibration unit 14-1 to a calibration unit 14-n, and respectively have a transmission unit 13-1 to a transmission unit 13-n. In the present example, the first gas sensor apparatus 100-1 to the first gas sensor apparatus 100-n respectively have a first gas sensor 11-1 to a first gas sensor 11-n. In FIG. 5, an internal configuration of the first gas sensor apparatus 100 is omitted.

A plurality of gas sensors 600 (a gas sensor 600-1 to a gas sensor 600-n) may be arranged in the measurement target 501. The gas sensor 600-1 to the gas sensor 600-n may respectively transmit calibration information Ic1 to calibration information Icn. The calibration information Ic1 to the calibration information Icn may be different from each other, or may be the same.

The calibration unit 14-1 may calibrate, based on the calibration information Ic1 to the calibration information Icn, the concentration of the gas 503 (see FIG. 1) of the measurement target 501 which is calculated based on the output of the first gas sensor 11-1. The calibration unit 14-2 may calibrate, based on the calibration information Ic1 to the calibration information Icn, the concentration of the gas 503 in the measurement target 501 which is calculated based on the output of the first gas sensor 11-2. Similarly, the calibration unit 14-n may calibrate, based on the calibration information Ic1 to the calibration information Icn, the concentration of the gas 503 in the measurement target 501 which is calculated based on the output of the first gas sensor 11-n.

In the present example, the calibration unit 14 of one first gas sensor apparatus 100 is configured to calibrate, based on the plurality of pieces of calibration information Ic, the concentration of the gas 503 (see FIG. 1) which is calculated based on the output of the first gas sensor 11 of the one first gas sensor apparatus 100. Thus, the output of the first gas sensor 11 is more likely to be calibrated to an accurate concentration as compared with a case where the output of the first gas sensor 11 is calibrated based on one piece of the calibration information Ic.

In the present example, the calibration unit 14-1 to the calibration unit 14-n respectively include a computation unit 120-1 to a computation unit 120-n. The computation unit 120-1 to the computation unit 120-n may respectively compute first calibration information Ic′1 to first calibration information Ic′n. The gas concentration calculated based on the outputs of the first gas sensor 11-1 to the first gas sensor 11-n may be respectively calibrated based on the first calibration information Ic′1 to the first calibration information Ic′n.

The transmission unit 13-1 to the transmission unit 13-n may respectively transmit the first calibration information Ic′1 to the first calibration information Ic′n. When the plurality of living bodies 90 (see FIG. 1) have moved to the measurement target 502, the transmission unit 13-1 to the transmission unit 13-n may respectively transmit the first calibration information Ic′1 to the first calibration information Ic′n to the interior space 508.

The reception unit 20 of the second gas sensor apparatus 200 may receive the first calibration information Ic′ in each of the plurality of first gas sensor apparatuses 100. In the present example, the reception unit 20 receives the first calibration information Ic′1 to the first calibration information Ic′n respectively transmitted by the transmission unit 13-1 to the transmission unit 13-n.

The calibration unit 24 of the second gas sensor apparatus 200 may calibrate, based on the plurality of first calibration information Ic′, the concentration the gas 504 (see FIG. 1) which is calculated based on the output of the second gas sensor 21. In the present example, the calibration unit 24 calibrates the concentration of the gas 504 based on the first calibration information Ic′1 to the first calibration information Ic′n. As a result, the output of the second gas sensor 21 is more likely to be calibrated to an accurate concentration as compared with a case where the output of the second gas sensor 21 is calibrated based on one piece of the first calibration information Ic′.

Reliability information indicating the calibration reliabilities of the first gas sensor 11-1 to the first gas sensor 11-n are respectively set as a calibration reliability R11 to a calibration reliability R1n. Reliability information indicating the calibration reliability R11 to the calibration reliability R1n are respectively set as reliability information Ir1-1 to reliability information Ir1-n. The transmission unit 13 (see FIG. 2 to FIG. 4) of each of the plurality of first gas sensor apparatuses 100 may transmit the reliability information Ir1 of each of the first gas sensor apparatuses 100. In the present example, the transmission unit 13-1 to the transmission unit 13-n respectively transmit the reliability information Ir1-1 to the reliability information Ir1-n.

The reception unit 20 of the second gas sensor apparatus 200 may receive the reliability information Ir1-1 to the reliability information Ir1-n. The calibration unit 24 may apply a weighting to the calibration reliability R11 to the calibration reliability R1n. The application of the weighting to the calibration reliability R11 to the calibration reliability R1n refers to a configuration where a weighting to the calibration reliability R1 with a high reliability is set to be greater than a weighting to the calibration reliability R1 with a low reliability. The calibration unit 24 may calibrate, based on the calibration reliability R1 to which the weighting has been applied, the concentration of the gas 504 (see FIG. 1) which is calculated based on the output of the second gas sensor 21.

The reliability information Ir1-1 to the reliability information Ir1-n may be stored in the storage unit 122. The computation unit 120 may compute a weighting of the calibration reliability R11 to the calibration reliability R1n based on the reliability information Ir1-1 to the reliability information Ir1-n which are stored in the storage unit 122. The computation unit 120 may compute the second calibration information Ic″ by applying the weighting to the calibration reliability R11 to the calibration reliability R1n. The calibration unit 24 may calibrate, based on the second calibration information Ic″, the concentration of the gas 504 (see FIG. 1) which is calculated based on the output of the second gas sensor 21. As a result, the concentration of the gas 504 is more likely to be calibrated to an accurate concentration as compared with a case where the concentration of the gas 504 is calibrated based on the second calibration information Ic″ to which no weighting of the calibration reliability R11 to the calibration reliability R1n has been applied.

When the reliability information Ir1 in which the calibration reliability R1 of the first gas sensor apparatus 100 is lower than the first threshold Rth1 is included in any of the reliability information Ir1-1 to the reliability information Ir1-n, the computation unit 120 may compute a weighting by eliminating the calibration reliability R1 that is lower than the first threshold Rth1. The computation by the computation unit 120 on a weighting by eliminating the calibration reliability R1 may refer to computation by the computation unit 120 to set a weighting of the calibration reliability R1 as zero.

The calibration unit 24 may calibrate, based on the highest calibration reliability R1 among the calibration reliability R11 to the calibration reliability R1n, the concentration of the gas 504 (see FIG. 1) which is calculated based on the output of the second gas sensor 21. The calibration unit 24 may calibrate the concentration of the gas 504 by setting a weighting of the highest calibration reliability R1 as 1 and setting a weighting of the other calibration reliabilities R1 as zero.

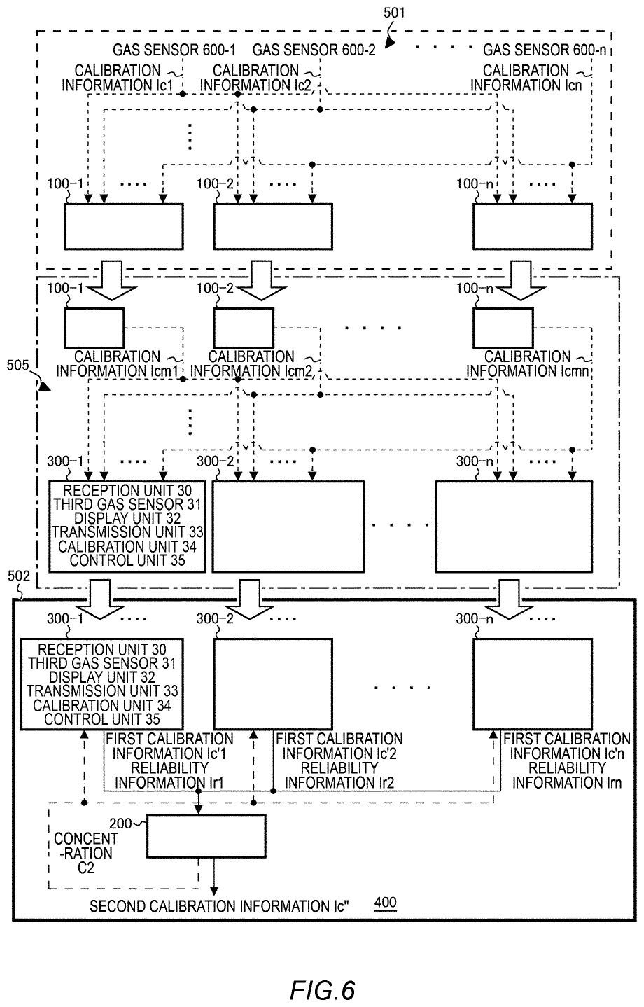

FIG. 6 illustrates an example of calibration of the gas concentration in the first gas sensor apparatus 100, the second gas sensor apparatus 200, and a third gas sensor apparatus 300 according to an embodiment of the present invention. The third gas sensor apparatus 300 may have a reception unit 30, a third gas sensor 31, a display unit 32, a transmission unit 33, a calibration unit 34, and a control unit 35. The block diagram of the third gas sensor apparatus 300 may be the same as the block diagrams of the first gas sensor apparatus 100 and the second gas sensor apparatus 200 which are illustrated in FIG. 2 to FIG. 4.

In the present example, the first gas sensor apparatus 100 moves from the measurement target 501 to a measurement target 505. The measurement target 505 may be outdoor, or may be indoor. When the measurement target 505 is outdoor, the gas concentration in the measurement target gas which is calculated based on the output of the first gas sensor 11 in the measurement target 501 and the gas concentration in the measurement target gas which is calculated based on the output of the first gas sensor 11 in the measurement target 505 may be the same or may be different from each other.

In the present example, the transmission unit 23 of the first gas sensor apparatus 100 in the measurement target 505 is configured to transmit first calibration information Icm. The transmission units 23 of the first gas sensor apparatus 100-1 to the first gas sensor apparatus 100-n may respectively transmit first calibration information Icm1 to first calibration information Icmn. In the present example, the reception unit 30 of the third gas sensor apparatus 300 is configured to receive the first calibration information Icm in the measurement target 505. The reception units 30 of the third gas sensor apparatus 300-1 to the third gas sensor apparatus 300-n may respectively receive the first calibration information Icm1 to the first calibration information Icmn.

In the present example, the calibration unit 34 of the third gas sensor apparatus 300 is configured to calibrate, based on the first calibration information Icm received by the reception unit 30, a concentration of a gas in the measurement target 505 which is calculated based on the output of the third gas sensor 31. The calibration units 34 of the third gas sensor apparatus 300-1 to the third gas sensor apparatus 300-n may respectively calibrate, based on the first calibration information Icm1 to the first calibration information Icmn, the concentration of the gas in the measurement target 505 which is calculated based on outputs of the respective third gas sensors 31 of the third gas sensor apparatus 300-1 to the third gas sensor apparatus 300-n.

In the present example, the third gas sensor apparatus 300 in which the concentration of the gas in the measurement target 505 has been calibrated moves from the measurement target 505 to the measurement target 502. In the present example, the transmission unit 33 of the third gas sensor apparatus 300 is configured to transmit the calibration information Ic′ of the third gas sensor apparatus 300. The transmission units 33 of the third gas sensor apparatus 300-1 to the third gas sensor apparatus 300-n may respectively transmit the calibration information Ic′1 to the calibration information Ic′n. In the present example, the reception unit 20 of the second gas sensor apparatus 200 is configured to receive the calibration information Ic′ in the measurement target 502. The reception unit 20 of the second gas sensor apparatus 200 may receive the calibration information Ic′1 to the calibration information Ic′n.

In the present example, the calibration unit 24 of the second gas sensor apparatus 200 is configured to calibrate, based on the calibration information Ic′ received by the reception unit 20, the concentration of the gas 504 (see FIG. 1) in the measurement target 502 which is calculated based on the output of the second gas sensor 21. In the present example, calibration information of a plurality of types (the calibration information Ic and the calibration information Icm) is to be reflected on the calibration information Ic′. Thus, the second gas sensor apparatus 200 is more likely to be accurately calibrated as compared with a case where calibration information of one type is to be reflected on the calibration information Ic′ (for example, the case of FIG. 5).

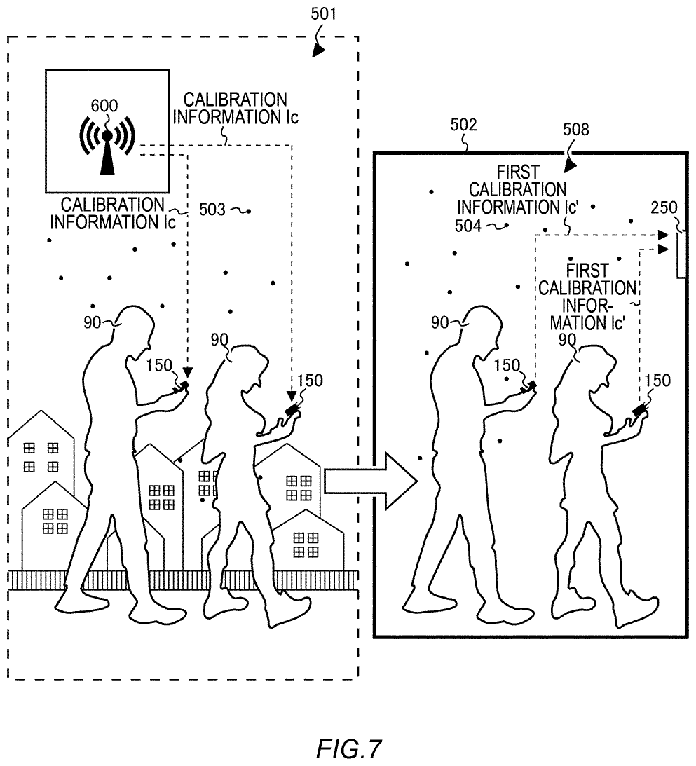

FIG. 7 illustrates an example of calibration of gas concentrations of a gas sensor apparatus 150 and a gas sensor apparatus 250 according to an embodiment of the present invention. The gas sensor apparatus 150 may be the same as the first gas sensor apparatus 100 illustrated in FIG. 1 to FIG. 6. The gas sensor apparatus 250 may be the same as the second gas sensor apparatus 200 illustrated in FIG. 1 to FIG. 6.

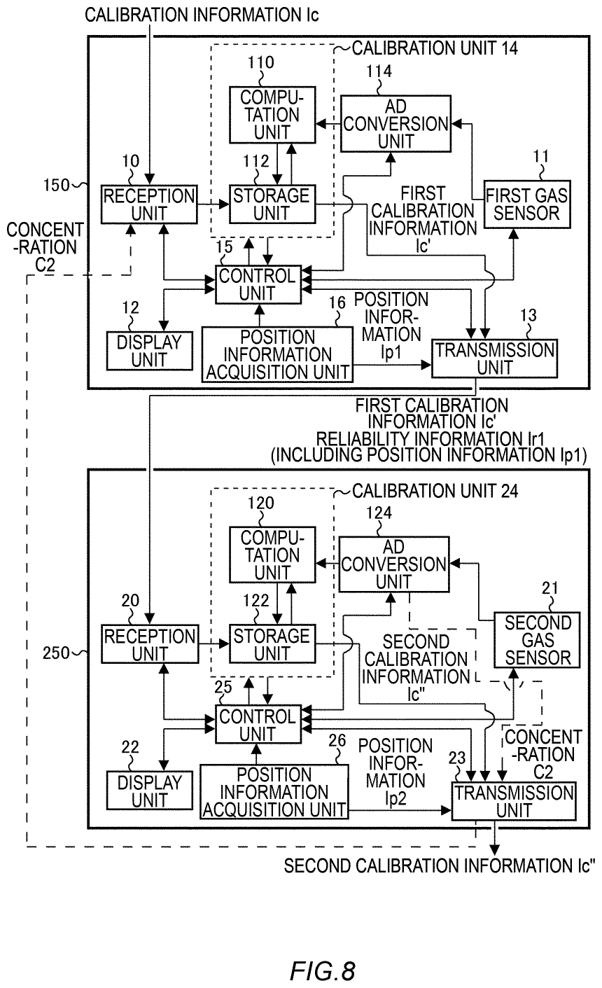

FIG. 8 is a block diagram illustrating an example of the gas sensor apparatus 150 and the gas sensor apparatus 250 according to an embodiment of the present invention. The gas sensor apparatus 150 may be the same as the first gas sensor apparatus 100 illustrated in FIG. 4. The gas sensor apparatus 250 may be the same as the second gas sensor apparatus 200 illustrated in FIG. 4.

The gas sensor apparatus 150 includes the transmission unit 13. The transmission unit 13 is configured to transmit the first calibration information Ic′ to the second gas sensor apparatus 250. The first calibration information Ic′ is calibration information for calibrating the concentration of the gas 503 (see FIG. 1) in the measurement target 501 (see FIG. 7) which is calculated based on the output of the first gas sensor 11. The first gas sensor apparatus 100 may be subjected to self-calibration, or may be calibrated based on another sensor (for example, the gas sensor 600 (see FIG. 7)).

The gas sensor apparatus 250 includes the reception unit 20 and the calibration unit 24. The reception unit 20 is configured to receive the first calibration information Ic′. The calibration unit 24 is configured to calibrate, based on the first calibration information Ic′ received by the reception unit 20, the concentration of the gas 504 (see FIG. 7) which is calculated based on the output of the second gas sensor 21.

The gas sensor apparatus 150 may include or may not include the first gas sensor 11. In the present example, the gas sensor apparatus 150 includes the first gas sensor 11. The gas sensor apparatus 250 may include or may not include the second gas sensor 21. In the present example, the gas sensor apparatus 250 includes the second gas sensor 21. When the gas sensor apparatus 150 is a mobile terminal, the first gas sensor 11 may be provided in the gas sensor apparatus 150. When the gas sensor apparatus 250 is a mobile terminal, the second gas sensor 21 may be provided in the gas sensor apparatus 250.

When the living body 90 (see FIG. 7) having the first gas sensor apparatus 150 remains in the measurement target 501, the transmission unit 13 may transmit the first calibration information Ic′ to an open space in the measurement target 501. When the living body 90 has moved to the measurement target 502 (see FIG. 1), the transmission unit 13 may transmit the first calibration information Ic′ to the interior space 508. The transmission unit 13 may wirelessly transmit the first calibration information Ic′.

Similarly as in the examples illustrated in FIG. 1 and FIG. 2, the measurement target 502 in which the concentration C1′ is calculated based on the output of the first gas sensor 11 and the measurement target 502 in which the concentration C2 is calculated based on the output of the second gas sensor 21 may be the same. A situation where the measurement target 502 in which the concentration C1′ is calculated and the measurement target 502 in which the concentration C2 is calculated are the same may refer to a situation where a type of the measurement target gas of the first gas sensor apparatus 150 and a type of the measurement target gas of the second gas sensor apparatus 250 are the same in the measurement target 502 as described above. The measurement target gas of the first gas sensor apparatus 150 and the second gas sensor apparatus 250 may be CO₂ (carbon dioxide), may be CH₄ methane, or may be alcohol.

A situation where the measurement target 502 in which the concentration C1′ is calculated is the same as the measurement target 502 in which the concentration C2 is calculated may refer to a situation where the first gas sensor 11 and the second gas sensor 21 share a same space (in the present example, the interior space 508 (see FIG. 7)) as described above. The same space may refer to at least one of a case where a temperature or a humidity in the space is the same, a case where an ID of short distance radio (Wi-Fi (registered trademark) or the like) is the same, or a case where, when the gas sensor apparatus 150 and the gas sensor apparatus 250 are a mobile terminal, an amplitude and a frequency of acoustic waves acquired by the mobile terminal are the same.

The second gas sensor 21 may be arranged in the interior space 508 (see FIG. 7). In the present example, the gas sensor apparatus 250 includes the second gas sensor 21, and the gas sensor apparatus 250 is arranged in the interior space 508.

The transmission unit 13 may transmit the reliability information Ir1 indicating the calibration reliability R1 of the first gas sensor apparatus 150 to the second gas sensor apparatus 250. The reception unit 20 may receive the reliability information Ir1. The calibration unit 24 may calibrate, based on the first calibration information Ic′ and the calibration reliability R1 of the first gas sensor apparatus 150, the concentration of the gas 504 (see FIG. 1) which is calculated based on the output of the second gas sensor 21. The calibration unit 24 may calibrate, based on the first calibration information Ic′, the concentration of the gas 504 (see FIG. 1) which is calculated based on the output of the second gas sensor 21 according to the reliability information Ir1.

When the reliability of the self-calibration of the second gas sensor apparatus 250 is equal to or lower than the second threshold Rth2 and also the calibration reliability R1 exceeds the first threshold Rth1, the calibration unit 24 may calibrate, based on the first calibration information Ic′, the concentration of the gas 504 (see FIG. 7) which is calculated based on the output of the second gas sensor 21. A case where the reliability of the self-calibration of the second gas sensor apparatus 250 is equal to or lower than the second threshold Rth2 may include a case where the second gas sensor apparatus 250 does not have a function of the self-calibration.

The calibration unit 24 may compare the calibration reliability R1 of the first gas sensor apparatus 150 which is received by the reception unit 20 with the calibration reliability R2 of the second gas sensor apparatus 250. The calibration reliability R2 of the second gas sensor apparatus 250 may include the calibration reliability R2 of the self-calibration of the second gas sensor apparatus 250 and the calibration reliability R2 in a case where the second gas sensor apparatus 250 is calibrated by another sensor.

When the calibration reliability R1 is higher than the calibration reliability R2, the calibration unit 24 may calibrate, based on the first calibration information Ic′ received by the reception unit 20, the concentration of the gas 504 (see FIG. 7) which is calculated based on the output of the second gas sensor 21. When the calibration reliability R1 is higher than the calibration reliability R2, since the concentration of the gas 504 is calibrated, the concentration of the gas 504 which is calculated based on the output of the second gas sensor 21 is likely to be accurate. When the calibration reliability R1 is equal to or lower than the calibration reliability R2, a configuration may be adopted where the calibration unit 24 does not calibrate the concentration of the gas 504 (see FIG. 7) which is calculated based on the output of the second gas sensor 21, or the calibration unit 24 may calibrate the concentration of the gas 504 based on the second calibration information Ic“. The second calibration information Ic” is calibration information for calibrating the concentration of the gas 504 (see FIG. 7) in the measurement target 502 (see FIG. 7) which is calculated based on the output of the second gas sensor 21.

The gas sensor apparatus 250 may further include the storage unit 122. The calibration reliability R1 of the first gas sensor apparatus 150 which is received by the reception unit 20 may be stored in the storage unit 122. The calibration reliability R1 stored in the storage unit 122 may refer to a past calibration reliability of the first gas sensor apparatus 150. The calibration unit 24 may compare the calibration reliability R1 stored in the storage unit 122 with a current calibration reliability R1 of the first gas sensor apparatus 150 which is received by the reception unit 20. When the current calibration reliability R1 is higher than the calibration reliability R1 which is stored in the storage unit 122, the calibration unit 24 may calibrate, based on the first calibration information Ic′, the concentration of the gas 504 (see FIG. 7) which is calculated based on the output of the second gas sensor 21.

When the calibration reliability R1 of the first gas sensor apparatus 150 which is received by the reception unit 20 is higher than the reference calibration reliability Cs, the calibration unit 24 may calibrate, based on the first calibration information Ic′, the gas concentration in the measurement target 502 which is calculated based on the output of the second gas sensor 21. The storage unit 122 may store the reference reliability information Cs. When the calibration reliability R1 of the first gas sensor apparatus 150 which is received by the reception unit 20 is higher than the reference calibration reliability Cs, the storage unit 122 may update the reference calibration reliability Cs based on the received calibration reliability R1 and store the updated reference calibration reliability Cs. The calibration unit 24 may calibrate, based on the first calibration information Ic′, the gas concentration in the measurement target 502 which is calculated based on the output of the second gas sensor 21 according to the updated reference reliability Cs.

When the calibration reliability R1 of the first gas sensor apparatus 150 which is received by the reception unit 20 is higher than the reference calibration reliability Cs, the storage unit 122 may update the reference calibration reliability Cs to the received calibration reliability R1 and store the updated reference calibration reliability Cs. When the calibration reliability R1 is lower than the reference calibration reliability Cs, a configuration may be adopted where the storage unit 122 does not update the reference calibration reliability Cs.

The reference reliability information Cs stored in the storage unit 122 may be the reference reliability information Cs updated based on the calibration reliability R1 or may be the calibration reliability R2. The calibration reliability R2 may include the calibration reliability R2 of the self-calibration of the second gas sensor apparatus 250 and the calibration reliability R2 in a case where the second gas sensor apparatus 200 is calibrated by another sensor.

The gas sensor apparatus 150 may further include the position information acquisition unit 16. The gas sensor apparatus 250 may further include the position information acquisition unit 26. The position information acquisition unit 16 is configured to acquire information of a position of the first gas sensor apparatus 150. The position information acquisition unit 26 is configured to acquire information of a position of the second gas sensor apparatus 250. The position information acquisition unit 16 and the position information acquisition unit 26 are, for example, a global positioning system (GPS).

The position information of the first gas sensor apparatus 150 is set as the position information Ip1. The reliability information Ir1 may include the position information Ip1. The position information of the second gas sensor apparatus 250 is set as the position information Ip2. The reliability information Ir2 may include the position information Ip2.

The calibration unit 24 may calibrate, based on the distance d between the position of the first gas sensor apparatus 150 and the position of the second gas sensor apparatus 250 and the first calibration information Ic′, the concentration of the gas 504 (see FIG. 1) which is calculated based on the output of the second gas sensor 21. As described above, the predetermined distance between the position of the first gas sensor 11 and the position of the second gas sensor 21 is the distance dp2. When the distance d is less than the distance dp2, the calibration unit 24 may calibrate the concentration of the gas 504 which is calculated based on the output of the second gas sensor 21. As described above, the distance dp2 may refer to a distance at which likelihood that the first gas sensor 11 and the second gas sensor 21 are arranged in a same space (for example, the interior space 508 of FIG. 7) is high.

When the distance d exceeds the distance dp2, a configuration may be adopted where the calibration unit 24 does not calibrate the concentration of the gas 504 which is calculated based on the output of the second gas sensor 21. The calibration unit 24 may calibrate, based on the first calibration information Ic′, the concentration of the gas 504 (see FIG. 1) which is calculated based on the output of the second gas sensor 21 according to the distance d.

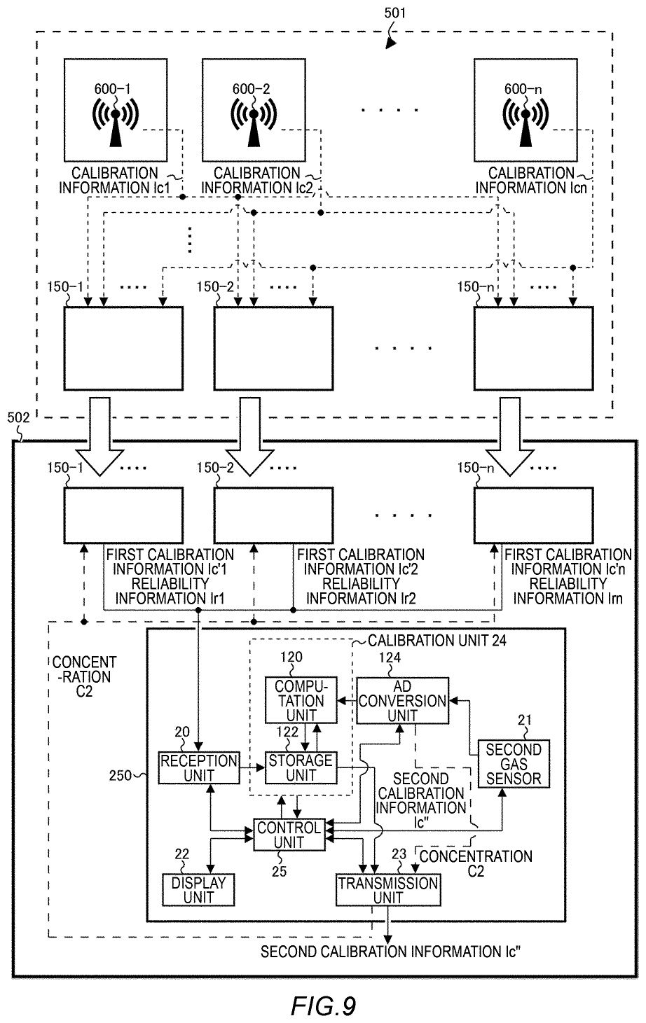

FIG. 9 is a block diagram illustrating another example of the gas sensor apparatus 150 and the gas sensor apparatus 250 according to an embodiment of the present invention. In the present example, a plurality of the gas sensor apparatuses 150 (a gas sensor apparatus 150-1 to a gas sensor apparatus 150-n) are arranged in the measurement target 501, and the single gas sensor apparatus 250 is arranged in the measurement target 502. The gas sensor apparatus 150 may be the same as the first gas sensor apparatus 100 illustrated in FIG. 5. The gas sensor apparatus 250 may be the same as the second gas sensor apparatus 200 illustrated in FIG. 5.

The transmission unit 13-1 to the transmission unit 13-n (see FIG. 8) respectively included in the gas sensor apparatus 150-1 to the gas sensor apparatus 150-n may respectively transmit the first calibration information Ic′1 to the first calibration information Ic′n. When the plurality of living bodies 90 (see FIG. 7) have moved to the measurement target 502, the transmission unit 13-1 to the transmission unit 13-n may respectively transmit the first calibration information Ic′1 to the first calibration information Ic′n to the interior space 508 (see FIG. 7).

The reception unit 20 of the gas sensor apparatus 250 may receive the first calibration information Ic′ of each of the plurality of gas sensor apparatuses 150. In the present example, the reception unit 20 receives the first calibration information Ic′1 to the first calibration information Ic′n respectively transmitted by the transmission unit 13-1 to the transmission unit 13-n (see FIG. 8).

The calibration unit 24 of the gas sensor apparatus 250 may calibrate, based on the plurality of first calibration information Ic′, the concentration of the gas 504 (see FIG. 7) which is calculated based on the output of the second gas sensor 21. In the present example, the calibration unit 24 calibrates the concentration of the gas 504 based on the first calibration information Ic′1 to the first calibration information Ic′n. As a result, the concentration of the gas 504 is more likely to be calibrated to an accurate concentration as compared with a case where the concentration of the gas 504 is calibrated based on one piece of the first calibration information Ic′.

The transmission unit 13 (see FIG. 8) of each of the plurality of gas sensor apparatuses 150 may transmit the reliability information Ir1 of each of the first gas sensor apparatuses 150. In the present example, the transmission unit 13-1 to the transmission unit 13-n respectively transmit the reliability information Ir1-1 to the reliability information Ir1-n.

The reception unit 20 may receive the reliability information Ir1 of each of the plurality of first gas sensor apparatuses 150. In the present example, the reception unit 20 receives the reliability information Ir1-1 to the reliability information Ir1-n. The reliability information Ir1-1 to the reliability information Ir1-n may be stored in the storage unit 122.

The calibration unit 24 may apply a weighting to the calibration reliability R11 to the calibration reliability R1n of each of the plurality of first gas sensor apparatuses 150. The calibration unit 24 may calibrate, based on the calibration reliability R1 to which the weighting has been applied, the concentration of the gas 504 (see FIG. 1) which is calculated based on the output of the second gas sensor 21. As a result, the concentration of the gas 504 is more likely to be calibrated to an accurate concentration as compared with a case where the concentration of the gas 504 is calibrated based on the calibration reliability R1 on which no weighting has been applied. The calibration unit 24 may calibrate, based on a highest calibration reliability R1 among the calibration reliability R11 to the calibration reliability R1n, the concentration of the gas 504 (see FIG. 1) which is calculated based on the output of the second gas sensor 21.

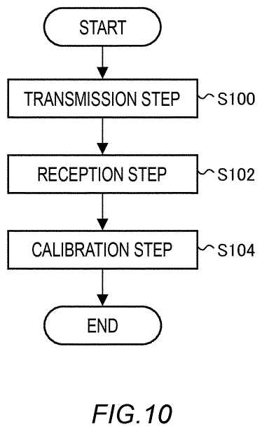

FIG. 10 is a flowchart illustrating an example of a gas sensor calibration method according to an embodiment of the present invention. The gas sensor calibration method according to an embodiment of the present invention will be described by using the gas sensor system 400 illustrated in FIG. 4 and FIG. 5 as an example.

Transmission step S100 is a step for the transmission unit 13 to transmit the first calibration information Ic′. The first calibration information Ic′ is calibration information for calibrating the concentration of the gas 503 (see FIG. 1) in the measurement target 501 (see FIG. 1) which is calculated based on the output of the first gas sensor 11.

Reception step S102 is a step for the reception unit 20 to receive the first calibration information Ic′ transmitted in the transmission step S100. Calibration step S104 is a step for the calibration unit 24 to calibrate, based on the first calibration information Ic′ received in the reception step S102, the concentration of the gas 504 (see FIG. 1) in the measurement target 502 (see FIG. 1) which is calculated based on the output of the second gas sensor 21.

The transmission step S100 may be a step for the transmission unit 13 to further transmit the reliability information Ir1 indicating the calibration reliability R1 of the first gas sensor apparatus 100. The reception step S102 may be a step for the reception unit 20 to further receive the reliability information Ir1 transmitted in the transmission step S100.

The calibration step S104 may be a step for the calibration unit 24 to calibrate, based on the first calibration information Ic′ and the reliability information Ir1, the gas concentration of the gas 504 in the measurement target 502 which is calculated based on the output of the second gas sensor 21. The calibration step S104 may be a step for the calibration unit 24 to calibrate, based on the first calibration information Ic′ received in the reception step S102, the concentration of the gas 504 which is calculated based on the output of the second gas sensor 21 according to the calibration reliability R1.

When the calibration reliability R2 of the self-calibration of the second gas sensor apparatus 200 is equal to or lower than the predetermined second threshold Rth2 and also the calibration reliability R1 exceeds the first threshold Rth1, the calibration step S104 may be a step for the calibration unit 24 to calibrate, based on the first calibration information Ic′ received in the reception step S102, the concentration of the gas 504 in the measurement target 502 which is calculated based on the output of the second gas sensor 21.

The calibration step S104 may be a step for the calibration unit 24 to compare the calibration reliability R1 received in the reception step S102 with the calibration reliability R2, and when the calibration reliability R1 is higher than the calibration reliability R2, to calibrate, based on the first calibration information Ic′ received in the reception step S102, the concentration of the gas 504 in the measurement target 502 which is calculated based on the output of the second gas sensor 21. When the calibration reliability R1 of the first gas sensor apparatus 100 which is received in the reception step S102 is higher than the reference calibration reliability Cs, the calibration step S104 may be a step for the calibration unit 24 to calibrate, based on the first calibration information Ic′, the gas concentration in the measurement target 502 which is calculated based on the output of the second gas sensor 21.

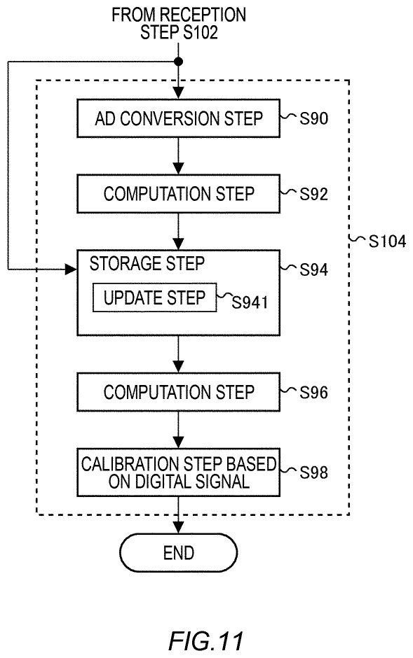

FIG. 11 illustrates an example of a detail of the calibration step S104 in FIG. 10. AD conversion step S90 is a step for the AD conversion unit 124 to convert an output of an analog signal of the second gas sensor 21 into a digital signal. Computation step S92 is a step for the computation unit 120 to calculate the concentration of the gas 504 based on the digital signal converted in the AD conversion step S90. Storage step S94 is a step for the storage unit 112 to store the concentration of the gas 504 which is calculated in the computation step S92. The storage step S94 may be a step of storing the correlation Cr′ between the concentration of the gas 504 which is calculated in the computation step S92 and the first calibration information Ic′.

Computation step S96 is a step for the computation unit 120 to compute the second calibration information Ic″ based on the first calibration information Ic′ received in the reception step S102 and the correlation Cr′ stored in the storage step S94. Calibration step S98 based on a digital signal is a step for the calibration unit 24 (see FIG. 4) to calibrate, by the second calibration information Ic″ of the digital signal, the concentration of the gas 504 (see FIG. 1) in the measurement target 502 (see FIG. 1) which is calculated based on the output of the second gas sensor 21. Note that the computation step S96 may be a step for the calibration unit 24 to perform calibration while calculating the concentration of the gas 504 based on the digital signal converted in the AD conversion step S90 and the first calibration information Ic′ received in the reception step S102.

When the living body 90 (see FIG. 1) having the first gas sensor apparatus 100 has moved from the measurement target 501 to the measurement target 502, the measurement target 502 in which the concentration of the gas 504 is calculated based on the output of the first gas sensor 11 may be the same as the measurement target 502 in which the concentration of the gas 504 is calculated based on the output of the second gas sensor 21. A situation where the measurement target 502 is the same may refer to a situation where a type of the measurement target gas of the first gas sensor apparatus 100 is the same as a type of the measurement target gas of the second gas sensor apparatus 200 in the measurement target 502. In the present example, the second gas sensor apparatus 200 is arranged in the interior space 508.

A situation where the measurement target 502 is the same may refer to a situation where the first gas sensor 11 and the second gas sensor 21 share a same space (in the present example, the interior space 508 (see FIG. 1)). The same space may refer to at least one of a case where a temperature or a humidity in the space is the same, a case where an ID of short distance radio (Wi-Fi (registered trademark) or the like) is the same, or a case where, when the first gas sensor apparatus 100 and the second gas sensor apparatus 200 are a mobile terminal, an amplitude and a frequency of acoustic waves acquired by the mobile terminal are the same.

The storage unit 122 may store the reference reliability information Cs. When the calibration reliability R1 of the first gas sensor apparatus 100 which is received in the reception step S102 is higher than the reference calibration reliability Cs, the storage step S94 may include update step S941 for the storage unit 122 to update the reference calibration reliability Cs based on the received calibration reliability R1. In the storage step S94, the storage unit 122 may store the updated reference calibration reliability Cs. The calibration step S104 may be a step for the calibration unit 24 to calibrate, based on the first calibration information Ic′, the gas concentration in the measurement target 502 which is calculated based on the output of the second gas sensor 21 according to the updated reference reliability Cs.

When the calibration reliability R1 of the first gas sensor apparatus 100 which is received in the reception step S102 is higher than the reference calibration reliability Cs, the update step S941 may be a step for the storage unit 122 to update the reference calibration reliability Cs to the calibration reliability R1. When the calibration reliability R1 is lower than the reference calibration reliability Cs, in the storage step S94, a configuration may be adopted where the storage unit 122 does not update the reference calibration reliability Cs.

The reference reliability information Cs stored in the storage step S94 may be the reference reliability information Cs updated based on the calibration reliability R1, or may be the calibration reliability R2. The calibration reliability R2 may include the calibration reliability R2 of the self-calibration of the second gas sensor apparatus 200 and the calibration reliability R2 in a case where the second gas sensor apparatus 200 is calibrated by another sensor.

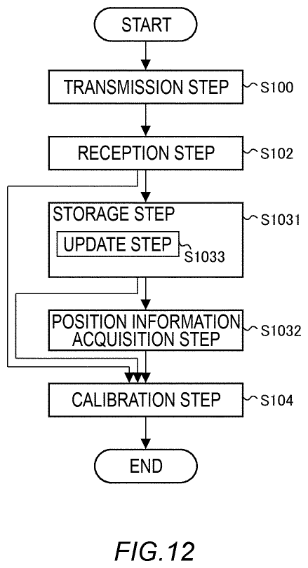

FIG. 12 is a flowchart illustrating another example of the gas sensor calibration method according to an embodiment of the present invention. The gas sensor calibration method of the present example is different from the gas sensor calibration method illustrated in FIG. 10 in that storage step S1031 and position information acquisition step S1032 are further included. The gas sensor calibration method of the present example will be described by using the gas sensor system 400 illustrated in FIG. 4 and FIG. 5 as an example.

The storage step S1031 is a step for the storage unit 122 to store the reliability information Ir1 received in the reception step S102. The calibration step S104 is a step for the calibration unit 24 to compare the reliability information Ir1 received in the reception step S102 with the reliability information Ir1 stored in the storage step S1031, and when the reliability information Ir1 received in the reception step S102 is higher than the reliability information Ir1 stored in the storage step S1031, to calibrate, based on the first calibration information Ic′ received in the reception step S102, the concentration of the gas 504 (see FIG. 1) in the measurement target 502 (see FIG. 1) which is calculated based on the output of the second gas sensor 21. The calibration step S104 may be performed after the storage step S1031.

When the calibration reliability R1 of the first gas sensor apparatus 100 which is received in the reception step S102 is higher than the reference calibration reliability Cs, the storage step S1031 may include update step S1033 for the storage unit 122 to update the reference calibration reliability Cs based on the received calibration reliability R1. In storage step S1031, the storage unit 122 may store the updated reference calibration reliability Cs. The calibration step S104 may be a step for the calibration unit 24 to calibrate, based on the first calibration information Ic′, the gas concentration in the measurement target 502 which is calculated based on the output of the second gas sensor 21 according to the updated reference reliability Cs.

When the calibration reliability R1 of the first gas sensor apparatus 100 which is received in the reception step S102 is higher than the reference calibration reliability Cs, the update step S1033 may be a step for the storage unit 122 to update the reference calibration reliability Cs to the calibration reliability R1. When the calibration reliability R1 is lower than the reference calibration reliability Cs, in the storage step S1031, a configuration may be adopted where the storage unit 122 does not update the reference calibration reliability Cs.

The reference reliability information Cs stored in the storage step S1031 may be the reference reliability information Cs updated based on the calibration reliability R1, or may be the calibration reliability R2. The calibration reliability R2 may include the calibration reliability R2 of the self-calibration of the second gas sensor apparatus 200 and the calibration reliability R2 in a case where the second gas sensor apparatus 200 is calibrated by another sensor.

The position information acquisition step S1032 is a step for the position information acquisition unit 26 to acquire the position information Ip2 of the second gas sensor apparatus 200. In the position information acquisition step S1032, the position information acquisition unit 16 may acquire the position information Ip1 of the first gas sensor apparatus 100. The position information acquisition unit 16 and the position information acquisition unit 26 are, for example, a global positioning system (GPS).

The reliability information Ir1 may include the position information Ip1. As described above, the distance d is the distance between the position of the first gas sensor apparatus 100 and the position of the second gas sensor apparatus 200 which is acquired by the position information acquisition unit 26. The calibration step S104 may be a step for the calibration unit 24 to calibrate, based on the distance d and the first calibration information Ic′, the concentration of the gas 504 (see FIG. 1) which is calculated based on the output of the second gas sensor 21. The calibration step S104 may be a step for the calibration unit 24 to calibrate, based on the first calibration information Ic′ received in the reception step S102, the concentration of the gas 504 which is calculated based on the output of the second gas sensor 21 according to the distance d. The calibration step S104 may be performed after the position information acquisition step S1032.

As described above, the distance dp2 is the predetermined distance between the position of the first gas sensor 11 and the position of the second gas sensor 21. When the distance d is less than the distance dp2, the calibration step S104 may be a step for the calibration unit 24 to calibrate the concentration of the gas 504 (see FIG. 1) which is calculated based on the output of the second gas sensor 21. In the calibration step S104, when the distance d is equal to or more than the distance dp2, a configuration may be adopted where the calibration unit 24 does not calibrate the concentration of the gas 504, or the calibration unit 24 may calibrate the concentration of the gas 504 based on the second calibration information Ic“. The second calibration information Ic” is calibration information for calibrating the concentration of the gas 504 (see FIG. 7) in the measurement target 502 (see FIG. 7) which is calculated based on the output of the second gas sensor 21.

An order of the storage step S1031 and the position information acquisition step S1032 may be reversed. That is, after the reception step S102, the position information acquisition step S1032 may be performed, and after the position information acquisition step S1032, the storage step S1031 may be performed. When the order of the storage step S1031 and the position information acquisition step S1032 is reversed, the storage unit 122 may store the position information Ip2, or may store the position information Ip2 and the position information Ip1.

The reception step S102 may be a step for the reception unit 20 to receive the first calibration information Ic′ in each of the plurality of first gas sensor apparatuses 100. The calibration step S104 may be a step for the calibration unit 24 to calibrate, based on each of the first calibration information Ic′ received in the reception step S102, the concentration of the gas 504 (see FIG. 1) which is calculated based on the output of the second gas sensor 21. In the present example, in the calibration step S104, the calibration unit 24 calibrates the concentration of the gas 504 based on the first calibration information Ic′1 to the first calibration information Ic′n. As a result, the concentration of the gas 504 is more likely to be calibrated to an accurate concentration as compared with a case where the concentration of the gas 504 is calibrated based on one piece of the first calibration information Ic′.

The transmission step S100 may be a step for the transmission unit 13 to further transmit the reliability information Ir1 of each of the plurality of first gas sensor apparatuses 100. The reception step S102 may be a step for the reception unit 20 to further receive the reliability information Ir1 transmitted in the transmission step S100. The calibration step S104 may be a step for the calibration unit 24 to apply a weighting to the calibration reliability R11 to the calibration reliability R1n, and to calibrate, based on the calibration reliability R1 to which the weighting has been applied, the concentration of the gas 504 (see FIG. 1) which is calculated based on the output of the second gas sensor 21.

The storage step S1031 may be a step of storing the reliability information Ir1 of each of the plurality of first gas sensor apparatuses 100 which is received in the reception step S102. The computation step S96 illustrated in FIG. 11 may be a step for the computation unit 120 to compute a weighting of the calibration reliability R11 to the calibration reliability R1n based on each of the reliability information Ir1 stored in the storage step S1031, and also to compute the second calibration information Ic″ by applying the weighting to the calibration reliability R11 to the calibration reliability R1n.

The calibration step S98 based on the digital signal which is illustrated in FIG. 11 may be a step for the calibration unit 24 (see FIG. 4 and FIG. 5) to calibrate the concentration of the gas 504 (see FIG. 1) in the measurement target 502 (see FIG. 1) which is calculated based on the output of the second gas sensor 21 by the digital signal of the second calibration information Ic″ to which the weighting has been applied. As a result, the concentration of the gas 504 is more likely to be calibrated to an accurate concentration as compared with a case where the concentration of the gas 504 is calibrated based on the second calibration information Ic″ to which no weighting of the calibration reliability R1 has been applied.

The calibration step S104 may be a step for the calibration unit 24 to calibrate, based on the highest calibration reliability R1 among the calibration reliability R11 to the calibration reliability R1n, the concentration of the gas 504 (see FIG. 1) which is calculated based on the output of the second gas sensor 21. In the calibration step S104, the calibration unit 24 may calibrate the concentration of the gas 504 by setting a weighting of the highest calibration reliability R1 as 1 and setting a weighting of the other calibration reliabilities R1 as zero.

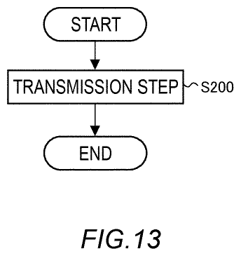

FIG. 13 is a flowchart illustrating an example of the gas sensor calibration method according to an embodiment of the present invention. The gas sensor calibration method according to an embodiment of the present invention will be described by using the gas sensor apparatus 150 illustrated in FIG. 8 and FIG. 9 as an example.

Transmission step S200 is a step for the transmission unit 13 to transmit the first calibration information Ic′ to the second gas sensor apparatus 250. The first calibration information Ic′ is calibration information for calibrating the concentration of the gas 503 (see FIG. 7) in the measurement target 501 (see FIG. 7) which is calculated based on the output of the first gas sensor 11. The transmission step S200 may be a step for the transmission unit 13 to further transmit the reliability information Ir1 indicating the calibration reliability R1 of the first gas sensor apparatus 150 to the second gas sensor apparatus 250.

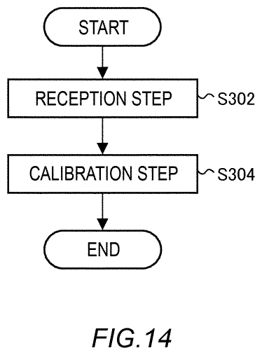

FIG. 14 is a flowchart illustrating an example of the gas sensor calibration method according to an embodiment of the present invention. The gas sensor calibration method according to an embodiment of the present invention will be described by using the gas sensor apparatus 250 illustrated in FIG. 8 and FIG. 9 as an example.

Reception step S302 is a step for the reception unit 20 to receive the first calibration information Ic′ of the first gas sensor apparatus 150. Calibration step S304 is a step for the calibration unit 24 to calibrate, based on the first calibration information Ic′ received in the reception step S102, the concentration of the gas 504 (see FIG. 7) in the measurement target 502 (see FIG. 7) which is calculated based on the output of the second gas sensor 21.

According to the gas sensor calibration method illustrated in FIG. 13 and FIG. 14, when the living body 90 (see FIG. 7) having the first gas sensor apparatus 150 has moved from the measurement target 501 to the measurement target 502, the measurement target 502 in which the concentration of the gas 504 is calculated based on the output of the first gas sensor 11 may be the same as the measurement target 502 in which the concentration of the gas 504 is calculated based on the output of the second gas sensor 21. A situation where the measurement target 502 is the same may refer to a situation where a type of the measurement target gas of the first gas sensor apparatus 150 is the same as a type of the measurement target gas of the second gas sensor apparatus 250 in the measurement target 502 as described above. In the present example, the second gas sensor apparatus 250 is arranged in the interior space 508.

A situation where the measurement target 502 is the same may refer to a situation where the first gas sensor 11 and the second gas sensor 21 share a same space (in the present example, the interior space 508 (see FIG. 7)) as described above. The same space may refer to at least one of a case where a temperature or a humidity in the space is the same, a case where an ID of short distance radio (Wi-Fi (registered trademark) or the like) is the same, or a case where, when the first gas sensor apparatus 150 and the second gas sensor apparatus 250 are a mobile terminal, an amplitude and a frequency of acoustic waves acquired by the mobile terminal are the same.

The reception step S302 may be a step for the reception unit 20 to further receive the reliability information Ir1 indicating the calibration reliability R1. The calibration step S304 may be a step for the calibration unit 24 to calibrate, based on the first calibration information Ic′ and the reliability information Ir1, the gas concentration of the gas 504 in the measurement target 502 which is calculated based on the output of the second gas sensor 21. The calibration step S304 may be a step for the calibration unit 24 to calibrate, based on the first calibration information Ic′ received in the reception step S302, the concentration of the gas 504 which is calculated based on the output of the second gas sensor 21 according to the calibration reliability R1.

When the calibration reliability R2 of the self-calibration of the second gas sensor apparatus 250 is equal to or lower than the predetermined second threshold Rth2 and also the calibration reliability R1 exceeds the first threshold Rth1, the calibration step S304 may be a step for the calibration unit 24 to calibrate, based on the first calibration information Ic′ received in the reception step S302, the concentration of the gas 504 in the measurement target 502 which is calculated based on the output of the second gas sensor 21.

The calibration step S304 may be a step for the calibration unit 24 to compare the calibration reliability R1 of the first gas sensor apparatus 150 with the calibration reliability R2, and when the calibration reliability R1 is higher than the calibration reliability R2, to calibrate, based on the first calibration information Ic′ received in the reception step S302, the concentration of the gas 504 in the measurement target 502 which is calculated based on the output of the second gas sensor 21. When the calibration reliability R1 of the first gas sensor apparatus 150 which is received in the reception step S302 is higher than the reference calibration reliability Cs, the calibration step S304 may be a step for the calibration unit 24 to calibrate, based on the first calibration information Ic′, the gas concentration in the measurement target 502 which is calculated based on the output of the second gas sensor 21.

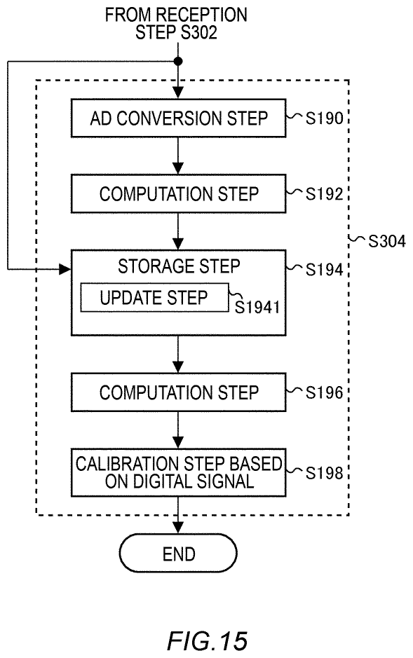

FIG. 15 illustrates an example of a detail of the calibration step S304 in FIG. 14. AD conversion step S190 is a step for the AD conversion unit 124 to convert an output of an analog signal of the second gas sensor 21 into a digital signal. Computation step S192 is a step for the computation unit 120 to calculate the concentration of the gas 504 based on the digital signal converted in the AD conversion step S190. Storage step S194 is a step for the storage unit 112 to store the concentration of the gas 504 which is calculated in the computation step S192. The storage step S194 may be a step of storing the correlation Cr′ between the concentration of the gas 504 which is calculated in the computation step S192 and the first calibration information Ic′.

Computation step S196 is a step for the computation unit 120 to compute the second calibration information Ic″ based on the first calibration information Ic′ received in the reception step S302 and the correlation Cr′ stored in the storage step S194. Calibration step S198 based on a digital signal is a step for the calibration unit 24 (see FIG. 4) to calibrate, based on the second calibration information Ic″ of the digital signal, the concentration of the gas 504 (see FIG. 7) in the measurement target 502 (see FIG. 7) which is calculated based on the output of the second gas sensor 21. Note that the computation step S196 may be a step for the calibration unit 24 to perform calibration while calculating the concentration of the gas 504 based on the digital signal converted in the AD conversion step S190 and the second calibration information Ic″ received in the reception step S302.

The storage unit 122 may store the reference reliability information Cs. When the calibration reliability R1 of the first gas sensor apparatus 150 which is received in the reception step S302 is higher than the reference calibration reliability Cs, the storage step S194 may include update step S1941 for the storage unit 122 to update the reference calibration reliability Cs based on the received calibration reliability R1. In the storage step S194, the storage unit 122 may store the updated reference calibration reliability Cs. The calibration step S304 may be a step for the calibration unit 24 to calibrate, based on the first calibration information Ic′, the gas concentration in the measurement target 502 which is calculated based on the output of the second gas sensor 21 according to the updated reference reliability Cs.

When the calibration reliability R1 of the first gas sensor apparatus 150 which is received in the reception step S302 is higher than the reference calibration reliability Cs, the update step S1941 may be a step for the storage unit 122 to update the reference calibration reliability Cs to the calibration reliability R1. In the storage step S194, when the calibration reliability R1 is lower than the reference calibration reliability Cs, a configuration may be adopted where the storage unit 122 does not update the reference calibration reliability Cs.

The reference reliability information Cs stored in the storage step S194 may be the reference reliability information Cs updated based on the calibration reliability R1 or may be the calibration reliability R2. The calibration reliability R2 may include the calibration reliability R2 of the self-calibration of the second gas sensor apparatus 250 and the calibration reliability R2 in a case where the second gas sensor apparatus 250 is calibrated by another sensor.

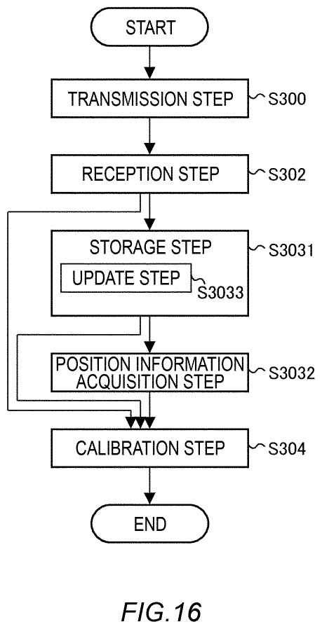

FIG. 16 is a flowchart illustrating another example of the gas sensor calibration method according to an embodiment of the present invention. The gas sensor calibration method of the present example is different from the gas sensor calibration method illustrated in FIG. 14 in that storage step S3031 and position information acquisition step S3032 are further included. The gas sensor calibration method of the present example will be described by using the gas sensor apparatus 250 illustrated in FIG. 8 and FIG. 9 as an example.

The storage step S3031 is a step for the storage unit 122 to store the reliability information Ir1 received in the reception step S302. The calibration step S304 is a step for the calibration unit 24 to compare the reliability information Ir1 received in the reception step S302 with the reliability information Ir1 stored in the storage step S3031, and when the reliability information Ir1 received in the reception step S302 is higher than the reliability information Ir1 stored in the storage step S3031, to calibrate, based on the first calibration information Ic′ received in the reception step S302, the concentration of the gas 504 (see FIG. 7) in the measurement target 502 (see FIG. 7) which is calculated based on the output of the second gas sensor 21. The calibration step S304 may be performed after the storage step S3031.

When the calibration reliability R1 of the first gas sensor apparatus 150 which is received in the reception step S102 is higher than the reference calibration reliability Cs, the storage step S3031 may include update step S3033 for the storage unit 122 to update the reference calibration reliability Cs based on the received calibration reliability R1. In the storage step S3031, the storage unit 122 may store the updated reference calibration reliability Cs. The calibration step S304 may be a step for the calibration unit 24 to calibrate, based on the first calibration information Ic′, the gas concentration in the measurement target 502 which is calculated based on the output of the second gas sensor 21 according to the updated reference reliability Cs.

When the calibration reliability R1 of the first gas sensor apparatus 150 which is received in the reception step S302 is higher than the reference calibration reliability Cs, the update step S3033 may be a step for the storage unit 122 to update the reference calibration reliability Cs to the calibration reliability R1. In the storage step S3031, when the calibration reliability R1 is lower than the reference calibration reliability Cs, a configuration may be adopted where the storage unit 122 does not update the reference calibration reliability Cs.

The reference reliability information Cs stored in the storage step S3031 may be the reference reliability information Cs updated based on the calibration reliability R1, or may be the calibration reliability R2. The calibration reliability R2 may include the calibration reliability R2 of the self-calibration of the second gas sensor apparatus 250 and the calibration reliability R2 in a case where the second gas sensor apparatus 250 is calibrated by another sensor.

The position information acquisition step S3032 is a step for the position information acquisition unit 26 to acquire the position information Ip2 of the second gas sensor apparatus 200. In the position information acquisition step S3032, the position information acquisition unit 16 may acquire the position information Ip1 of the first gas sensor apparatus 100. The position information acquisition unit 16 and the position information acquisition unit 26 are, for example, a global positioning system (GPS).

The reliability information Ir1 may include the position information Ip1. As described above, the distance d is the distance between the position of the first gas sensor apparatus 100 and the position of the second gas sensor apparatus 200 which is acquired by the position information acquisition unit 26. The calibration step S304 may be a step for the calibration unit 24 to calibrate, based on the distance d and the first calibration information Ic′, the concentration of the gas 504 (see FIG. 7) which is calculated based on the output of the second gas sensor 21. The calibration step S304 may be a step for the calibration unit 24 to calibrate, based on the first calibration information Ic′ received in the reception step S302, the concentration of the gas 504 which is calculated based on the output of the second gas sensor 21 according to the distance d. The calibration step S304 may be performed after the position information acquisition step S3032.

As described above, the distance dp2 is the predetermined distance between the position of the first gas sensor 11 and the position of the second gas sensor 21. When the distance d is less than the distance dp2, the calibration step S304 may be a step for the calibration unit 24 to calibrate the concentration of the gas 504 (see FIG. 7) which is calculated based on the output of the second gas sensor 21. In the calibration step S304, when the distance d is equal to or more than the distance dp2, a configuration may be adopted where the calibration unit 24 does not calibrate the concentration of the gas 504.

An order of the storage step S3031 and the position information acquisition step S3032 may be reversed. That is, after the reception step S302, the position information acquisition step S3032 may be performed, and after the position information acquisition step S3032, the storage step S3031 may be performed. When the order of the storage step S3031 and the position information acquisition step S3032 is reversed, the storage unit 122 may store the position information Ip2, or may store the position information Ip2 and the position information Ip1.

The reception step S302 may be a step for the reception unit 20 to receive the first calibration information Ic′ in each of the plurality of first gas sensor apparatuses 100. The calibration step S304 may be a step for the calibration unit 24 to calibrate, based on each of the first calibration information Ic′ received in the reception step S302, the concentration of the gas 504 (see FIG. 7) which is calculated based on the output of the second gas sensor 21. In the present example, in the calibration step S304, the calibration unit 24 calibrates the concentration of the gas 504 based on the first calibration information Ic′1 to the first calibration information Ic′n. As a result, the concentration of the gas 504 is more likely to be calibrated to an accurate concentration as compared with a case where the concentration of the gas 504 is calibrated based on one piece of the first calibration information Ic′.

The reception step S302 may be a step for the reception unit 20 to further receive the reliability information Ir1 of each of the plurality of first gas sensor apparatuses 100. The calibration step S304 may be a step for the calibration unit 24 to apply a weighting to the calibration reliability R11 to the calibration reliability R1n, and to calibrate, based on the calibration reliability R1 to which the weighting has been applied, the concentration of the gas 504 (see FIG. 7) which is calculated based on the output of the second gas sensor 21.

The storage step S3031 may be a step of storing the reliability information Ir1 of each of the plurality of first gas sensor apparatuses 100 which is received in the reception step S302. The computation step S196 illustrated in FIG. 15 may be a step for the computation unit 120 to compute a weighting of the calibration reliability R11 to the calibration reliability R1n based on each of the reliability information Ir1 stored in the storage step S3031, and also to compute the second calibration information Ic″ by applying the weighting to the calibration reliability R11 to the calibration reliability R1n.

The calibration step S198 based on the digital signal which is illustrated in FIG. 15 may be a step for the calibration unit 24 (see FIG. 8 and FIG. 9) to calibrate the concentration of the gas 504 (see FIG. 7) in the measurement target 502 (see FIG. 7) which is calculated based on the output of the second gas sensor 21 by the digital signal of the second calibration information Ic″ to which the weighting has been applied. As a result, the concentration of the gas 504 is more likely to be calibrated to an accurate concentration as compared with a case where the concentration of the gas 504 is calibrated based on the second calibration information Ic″ to which no weighting of the calibration reliability R1 has been applied.

The calibration step S304 may be a step for the calibration unit 24 to calibrate, based on the highest calibration reliability R1 among the calibration reliability R11 to the calibration reliability R1n, the concentration of the gas 504 (see FIG. 7) which is calculated based on the output of the second gas sensor 21. In the calibration step S304, the calibration unit 24 may calibrate the concentration of the gas 504 by setting a weighting of the highest calibration reliability R1 as 1 and setting a weighting of the other calibration reliabilities R1 as zero.

Various embodiments of the present invention may be described with reference to flowcharts and block diagrams. According to the various embodiments of the present invention, a block may represent (1) a step of a process where operations are executed or (2) a section of an apparatus having a role for executing operations.

A specific step may be executed by a dedicated circuit, a programmable circuit, or a processor. A specific section may be implemented by a dedicated circuit, a programmable circuit, or a processor. The programmable circuit and the processor may be supplied together with a computer-readable instruction. The computer-readable instruction may be stored on a computer-readable medium.

The dedicated circuit may include at least one of a digital hardware circuit or an analog hardware circuit. The dedicated circuit may include at least one of an integrated circuit (IC) or a discrete circuit. The programmable circuit may include a hardware circuit including logical AND, logical OR, logical XOR, logical NAND, logical NOR, or other logical operations. The programmable circuit may include a reconfigurable hardware circuit including a flip-flop, a register, a memory element such as a field programmable gate array (FPGA) and a programmable logic array (PLA), and the like.

Computer-readable media may include any tangible device that can store instructions for execution by a suitable device. Since the computer-readable medium includes the tangible device, the computer-readable medium having the instruction stored on the device constitutes a product including an instruction that may be executed in order to provide means to execute an operation specified by a flowchart or a block diagram.

The computer-readable medium may be, for example, an electronic storage medium, a magnetic storage medium, an optical storage medium, an electromagnetic storage medium, a semiconductor storage medium, or the like. More specifically, for example, the computer-readable medium may be a floppy disk, a diskette, a hard disk, a random access memory (RAM), a read only memory (ROM), an erasable programmable read only memory (EPROM or flash memory), an electrically erasable programmable read only memory (EEPROM), a static random access memory (SRAM), a compact disk read only memory (CD-ROM), a digital versatile disk (DVD), a Blu-ray (registered trademark) disk, a memory stick, an integrated circuit card, or the like.

The computer-readable instruction may include any of an assembler instruction, an instruction-set-architecture (ISA) instruction, a machine instruction, a machine dependent instruction, a microcode, a firmware instruction, state-setting data, a source code, and an object code. The source code and the object code may be written in any combination of one or more programming languages including an object-oriented programming language and a procedural programming language in related art. The object-oriented programming language may be, for example, Smalltalk (registered trademark), JAVA (registered trademark), C++, or the like. The procedural programming language may be, for example, a “C” programming language.

The computer-readable instruction may be provided to a general purpose computer, a special purpose computer, or a processor or a programmable circuit of another programmable data processing apparatus locally or via a local area network (LAN) or a wide area network (WAN) such as the Internet. A processor or programmable circuitry of a general purpose computer, a special purpose computer, or another programmable data processing apparatus may perform a computer-readable instruction in order to create means configured to perform operations designated by the flowchart illustrated in FIG. 10 to FIG. 16 or the block diagram illustrated in FIG. 2 to FIG. 5, FIG. 8, or FIG. 9. The processor may be, for example, a computer processor, a processing unit, a microprocessor, a digital signal processor, a controller, a microcontroller, or the like.

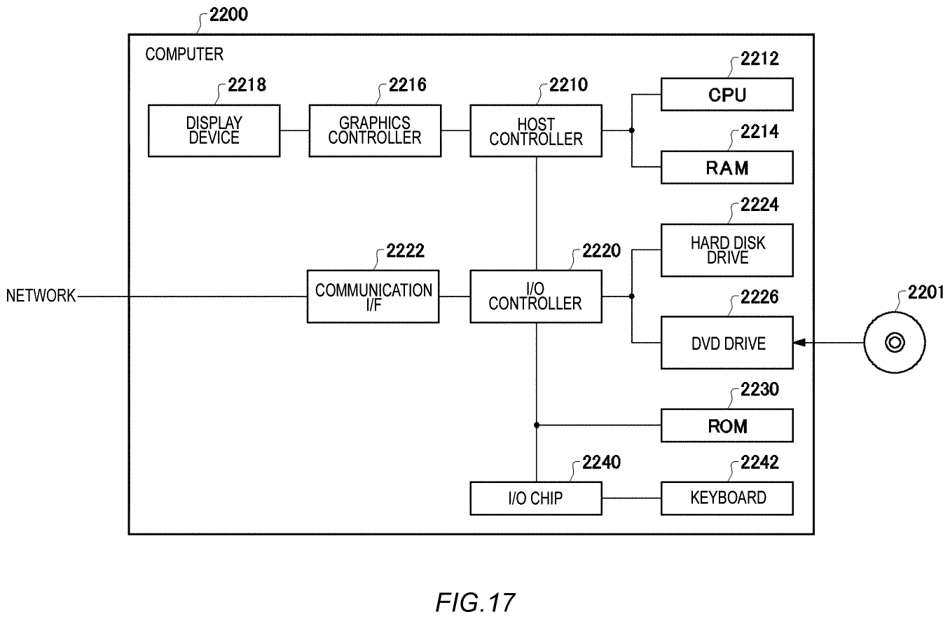

FIG. 17 illustrates an example of a computer 2200 in which the gas sensor system 400, the gas sensor apparatus 150, or the gas sensor apparatus 250 according to an embodiment of the present invention may be entirely or partially embodied. A program installed in the computer 2200 can cause the computer 2200 to perform operations associated with the gas sensor apparatus 150 or the gas sensor apparatus 250 according to an embodiment of the present invention or function as one or more sections of the gas sensor apparatus 150 or the gas sensor apparatus 250 or to perform the operations or the one or more sections, or can cause the computer 2200 to perform each of steps (see FIG. 10 to FIG. 16) according to the gas sensor calibration method of the present invention. The program may be executed by a CPU 2212 in order to cause the computer 2200 to perform particular operations associated with the flowcharts (FIG. 10 to FIG. 16) and some or all of blocks in the block diagrams (FIG. 2 to FIG. 5, FIG. 8, or FIG. 9) which are described in the present specification.

The computer 2200 according to an embodiment of the present invention includes the CPU 2212, a RAM 2214, a graphics controller 2216, and a display device 2218. The CPU 2212, the RAM 2214, the graphics controller 2216, and the display device 2218 are mutually connected by a host controller 2210. The computer 2200 further includes input and output units such as a communication interface 2222, a hard disk drive 2224, a DVD-ROM drive 2226, and an IC card drive. The communication interface 2222, the hard disk drive 2224, the DVD-ROM drive 2226, and the IC card drive, and the like are connected to the host controller 2210 via an input and output controller 2220. The computer further includes legacy input and output units such as a ROM 2230 and a keyboard 2242. The ROM 2230, the keyboard 2242, and the like are connected to the input and output controller 2220 through an input and output chip 2240.

The CPU 2212 is configured to operate according to programs stored in the ROM 2230 and the RAM 2214, thereby controlling each unit. The graphics controller 2216 is configured to obtain image data generated by the CPU 2212 on a frame buffer or the like provided in the RAM 2214 or in the RAM 2214 itself to cause the image data to be displayed on the display device 2218.

The communication interface 2222 is configured to communicate with other electronic devices via a network. The hard disk drive 2224 is configured to store programs and data used by the CPU 2212 within the computer 2200. The DVD-ROM drive 2226 is configured to read the programs or the data from the DVD-ROM 2201, and provide the read programs or data to the hard disk drive 2224 via the RAM 2214. The IC card drive is configured to read programs and data from an IC card, or write programs and data to the IC card.

The ROM 2230 is configured to store a boot program or the like executed by the computer 2200 at the time of activation, or a program depending on the hardware of the computer 2200. The input and output chip 2240 may connect various input and output units via a parallel port, a serial port, a keyboard port, a mouse port, or the like to the input and output controller 2220.

A program is provided by computer-readable media such as the DVD-ROM 2201 or the IC card. The program is read from the computer-readable media, installed into the hard disk drive 2224, RAM 2214, or ROM 2230, which are also examples of computer-readable media, and executed by the CPU 2212. The information processing described in these programs is read into the computer 2200, resulting in cooperation between a program and the above described various types of hardware resources. An apparatus or method may be constituted by realizing the operation or processing of information in accordance with the usage of the computer 2200.

For example, when a communication is performed between the computer 2200 and an external device, the CPU 2212 may execute a communication program loaded onto the RAM 2214 to instruct the communication interface 2222 to perform communication processing based on the processing described in the communication program. The communication interface 2222, under control of the CPU 2212, reads transmission data stored on a transmission buffering region provided in a recording medium such as the RAM 2214, the hard disk drive 2224, the DVD-ROM 2201, or the IC card, and transmits the read transmission data to a network or writes reception data received from a network to a reception buffering region or the like provided on the recording medium.

The CPU 2212 may cause all or a necessary portion of a file or a database to be read into the RAM 2214, the file or the database having been stored in an external recording medium such as the hard disk drive 2224, the DVD-ROM drive 2226 (DVD-ROM 2201), the IC card, or the like. The CPU 2212 may perform various types of processing on the data on the RAM 2214. The CPU 2212 may then write back the processed data to the external recording medium.

Various types of information such as various types of programs, data, tables, and databases may be stored in the recording medium to undergo information processing. The CPU 2212 may perform various types of processing on the data read from the RAM 2214, which includes various types of operations, processing of information, condition judging, conditional branch, unconditional branch, search or replace of information, or the like, as described throughout the present disclosure and designated by an instruction sequence of programs. The CPU 2212 may write the result back to the RAM 2214.

The CPU 2212 may search for information in a file, a database, or the like in the recording medium. For example, when a plurality of entries, each having an attribute value of a first attribute associated with an attribute value of a second attribute, are stored in the recording medium, the CPU 2212 may search for an entry matching the condition whose attribute value of the first attribute is designated, from among the plurality of entries, read the attribute value of the second attribute stored in the entry, and read a second attribute value to obtain the attribute value of the second attribute associated with the first attribute satisfying the predetermined condition.

The above explained program or software modules may be stored in the computer-readable media on the computer 2200 or of the computer 2200. A recording medium such as a hard disk or a RAM provided in a server system connected to a dedicated communication network or the Internet can be used as the computer-readable media. The program may be provided to the computer 2200 by the recording medium.

While the embodiments of the present invention have been described, the technical scope of the present invention is not limited to the above described embodiments. It is apparent to persons skilled in the art that various alterations and improvements can be added to the above described embodiments. It is also apparent from the scope of the claims that the embodiments added with such alterations or improvements can be included in the technical scope of the present invention.

Note that the operations, procedures, steps, and stages of each process performed by an apparatus, system, program, and method shown in the claims, embodiments, or diagrams can be performed in any order as long as the order is not indicated by “prior to,” “before,” or the like and as long as the output from a previous process is not used in a later process. Even if the process flow is described using phrases such as “first” or “next” in the claims, embodiments, or diagrams, it does not necessarily mean that the process must be performed in this order.

### Item 1

A gas sensor system comprising:

a first gas sensor apparatus including a transmission unit configured to transmit first calibration information for calibrating a gas concentration in a measurement target which is calculated based on an output of a first gas sensor; and

a second gas sensor apparatus including a reception unit configured to receive the first calibration information transmitted by the transmission unit, and a calibration unit configured to calibrate, based on the first calibration information received by the reception unit, a gas concentration in a measurement target which is calculated based on an output of a second gas sensor.

### Item 2

The gas sensor system according to item 1, wherein the measurement target in which the gas concentration is calculated based on the output of the first gas sensor is the same as the measurement target in which the gas concentration is calculated based on the output of the second gas sensor.

### Item 3

The gas sensor system according to item 1 or 2, wherein

the first gas sensor apparatus includes the first gas sensor, and

the first gas sensor apparatus is a mobile terminal.

### Item 4

The gas sensor system according to item 3, wherein the second gas sensor apparatus is arranged in an interior space in the measurement target in which the gas concentration is calculated based on the output of the second gas sensor.

### Item 5

The gas sensor system according to any one of items 1 to 4, wherein

the second gas sensor apparatus includes the second gas sensor, and

the second gas sensor apparatus is a mobile terminal.

### Item 6

The gas sensor system according to any one of items 1 to 5, wherein

the transmission unit is configured to further transmit reliability information indicating a calibration reliability of the first gas sensor apparatus,

the reception unit is configured to further receive the reliability information, and

the calibration unit is configured to calibrate, based on the received first calibration information, the gas concentration in the measurement target which is calculated based on the output of the second gas sensor according to the calibration reliability of the first gas sensor apparatus.

### Item 7

The gas sensor system according to item 6, wherein when a reliability of self-calibration of the second gas sensor apparatus is equal to or lower than a predetermined second threshold and also the calibration reliability of the first gas sensor apparatus exceeds a first threshold, the calibration unit is configured to calibrate, based on the received first calibration information, the gas concentration in the measurement target which is calculated based on the output of the second gas sensor.

### Item 8

The gas sensor system according to item 6 or 7, wherein the calibration unit is configured to compare the calibration reliability of the first gas sensor apparatus which is received by the reception unit with a calibration reliability of the second gas sensor apparatus, and when the calibration reliability of the first gas sensor apparatus is higher than the calibration reliability of the second gas sensor apparatus, to calibrate, based on the received first calibration information, the gas concentration in the measurement target which is calculated based on the output of the second gas sensor.

### Item 9

The gas sensor system according to any one of items 6 to 8, wherein

the second gas sensor apparatus further includes a storage unit configured to store the reliability information received by the reception unit, and

the calibration unit is configured to compare the calibration reliability of the first gas sensor apparatus which is received by the reception unit with the calibration reliability of the first gas sensor apparatus which is stored in the storage unit, and when the calibration reliability of the first gas sensor apparatus which is received by the reception unit is higher than the calibration reliability stored in the storage unit, to calibrate, based on the received first calibration information, the gas concentration in the measurement target which is calculated based on the output of the second gas sensor.

### Item 10

The gas sensor system according to any one of items 6 to 9, wherein

the second gas sensor apparatus further includes a position information acquisition unit configured to acquire information of a position of the second gas sensor apparatus,

the reliability information includes information of a position of the first gas sensor apparatus, and

the calibration unit is configured to calibrate, based on the received first calibration information, the gas concentration in the measurement target which is calculated based on the output of the second gas sensor according to a distance between the position of the first gas sensor apparatus and the position of the second gas sensor apparatus which is acquired by the position information acquisition unit.

### Item 11

The gas sensor system according to item 10, wherein when the distance is less than a predetermined distance, the calibration unit is configured to calibrate, based on the received first calibration information, the gas concentration in the measurement target which is calculated based on the output of the second gas sensor.

### Item 12

The gas sensor system according to any one of items 6 to 11, comprising:

a plurality of the first gas sensor apparatuses, wherein

the reception unit is configured to receive first calibration information of each of the plurality of first gas sensor apparatuses, and

the calibration unit is configured to calibrate, based on the first calibration information of each of the plurality of first gas sensor apparatuses, the gas concentration in the measurement target which is calculated based on the output of the second gas sensor.

### Item 13

The gas sensor system according to item 12, wherein

the transmission unit of each of the plurality of first gas sensor apparatuses is configured to transmit the reliability information of each of the first gas sensor apparatuses,

the reception unit is configured to receive the reliability information of each of the plurality of first gas sensor apparatuses, and

the calibration unit is configured to apply a weighting to the calibration reliability of each of the plurality of first gas sensor apparatuses, and to calibrate, based on the calibration reliability to which the weighting has been applied, the gas concentration in the measurement target which is calculated based on the output of the second gas sensor.

### Item 14

The gas sensor system according to item 13, wherein the calibration unit is configured to calibrate, based on a highest calibration reliability among the calibration reliabilities of each of the plurality of first gas sensor apparatuses, the gas concentration in the measurement target which is calculated based on the output of the second gas sensor.

### Item 15

A gas sensor apparatus comprising:

a transmission unit configured to transmit first calibration information to a second gas sensor apparatus, wherein

the first calibration information is calibration information for calibrating a gas concentration in a measurement target which is calculated based on an output of a first gas sensor.

### Item 16

The gas sensor apparatus according to item 15, wherein

the second gas sensor apparatus includes a second gas sensor, and

the measurement target in which the gas concentration is calculated based on the output of the first gas sensor is the same as the measurement target in which a gas concentration is calculated based on an output of the second gas sensor.

### Item 17

The gas sensor apparatus according to item 16, wherein the second gas sensor is provided in a mobile terminal.

### Item 18

The gas sensor apparatus according to any one of items 15 to 17, wherein

the first gas sensor is provided in a first gas sensor apparatus, and

the transmission unit is configured to further transmit reliability information indicating a calibration reliability of the first gas sensor apparatus to the second gas sensor apparatus.

### Item 19

A gas sensor apparatus comprising:

a reception unit configured to receive first calibration information of a first gas sensor apparatus; and

a calibration unit configured to calibrate, based on the first calibration information received by the reception unit, a gas concentration in a measurement target which is calculated based on an output of a second gas sensor.

### Item 20

The gas sensor apparatus according to item 19, wherein

the first gas sensor apparatus includes a first gas sensor, and

a measurement target in which a gas concentration is calculated based on an output of the first gas sensor is the same as the measurement target in which the gas concentration is calculated based on the output of the second gas sensor.

### Item 21

The gas sensor apparatus according to item 20, wherein the first gas sensor is provided in a mobile terminal.

### Item 22

The gas sensor apparatus according to item 21, wherein the second gas sensor is arranged in an interior space in the measurement target in which the gas concentration is calculated based on the output of the second gas sensor.

### Item 23

The gas sensor apparatus according to any one of items 19 to 22, wherein

the reception unit is configured to further receive reliability information indicating a calibration reliability of the first gas sensor apparatus, and

the calibration unit is configured to calibrate, based on the received first calibration information, the gas concentration in the measurement target which is calculated based on the output of the second gas sensor according to the calibration reliability of the first gas sensor apparatus.

### Item 24

The gas sensor apparatus according to item 23, wherein

the second gas sensor is provided in a second gas sensor apparatus, and

when a reliability of self-calibration of the second gas sensor apparatus is equal to or lower than a predetermined second threshold and also the calibration reliability of the first gas sensor apparatus exceeds a first threshold, the calibration unit is configured to calibrate, based on the received first calibration information, the gas concentration in the measurement target which is calculated based on the output of the second gas sensor.

### Item 25

The gas sensor apparatus according to item 24, wherein the calibration unit is configured to compare the calibration reliability of the first gas sensor apparatus which is received by the reception unit with a calibration reliability of the second gas sensor apparatus, and when the calibration reliability of the first gas sensor apparatus is higher than the calibration reliability of the second gas sensor apparatus, to calibrate, based on the received first calibration information, the gas concentration in the measurement target which is calculated based on the output of the second gas sensor.

### Item 26

The gas sensor apparatus according to item 24 or 25, further comprising:

a storage unit configured to store the reliability information received by the reception unit, wherein

the calibration unit is configured to compare the calibration reliability of the first gas sensor apparatus which is received by the reception unit with the calibration reliability of the first gas sensor apparatus which is stored in the storage unit, and when the calibration reliability of the first gas sensor apparatus which is received by the reception unit is higher than the calibration reliability stored in the storage unit, to calibrate, based on the received first calibration information, the gas concentration in the measurement target which is calculated based on the output of the second gas sensor.

### Item 27

The gas sensor apparatus according to any one of items 24 to 26, further comprising:

a position information acquisition unit configured to acquire information of a position of the second gas sensor apparatus, wherein

the reliability information includes information of a position of the first gas sensor apparatus, and

the calibration unit is configured to calibrate, based on the received first calibration information, the gas concentration in the measurement target which is calculated based on the output of the second gas sensor according to a distance between the position of the first gas sensor apparatus and the position of the second gas sensor apparatus which is acquired by the position information acquisition unit.

### Item 28

The gas sensor apparatus according to item 27, wherein when the distance is less than a predetermined distance, the calibration unit is configured to calibrate the gas concentration in the measurement target which is calculated based on the output of the second gas sensor.

### Item 29

The gas sensor apparatus according to any one of items 23 to 28, wherein

the reception unit is configured to receive the first calibration information of each of the plurality of first gas sensor apparatuses, and

the calibration unit is configured to calibrate, based on the first calibration information of each of the plurality of first gas sensor apparatuses, the gas concentration in the measurement target which is calculated based on the output of the second gas sensor.

### Item 30

The gas sensor apparatus according to item 29, wherein

the reception unit is configured to receive reliability information of each of the plurality of first gas sensor apparatuses, and

the calibration unit is configured to apply a weighting to the calibration reliability of each of the plurality of first gas sensor apparatuses, and to calibrate, based on the calibration reliability to which the weighting has been applied, the gas concentration in the measurement target which is calculated based on the output of the second gas sensor.

### Item 31

The gas sensor apparatus according to item 30, wherein the calibration unit is configured to calibrate, based on a highest calibration reliability among the calibration reliabilities of each of the plurality of first gas sensor apparatuses, the gas concentration in the measurement target which is calculated based on the output of the second gas sensor.

### Item 32

A gas sensor calibration method comprising:

transmitting, by a transmission unit, first calibration information, wherein the first calibration information is calibration information for calibrating a gas concentration in a measurement target which is calculated based on an output of a first gas sensor;

receiving, by a reception unit, the first calibration information transmitted in the transmitting; and

calibrating, by a calibration unit, based on the first calibration information received in the receiving, a gas concentration in a measurement target which is calculated based on an output of a second gas sensor.

### Item 33

The gas sensor calibration method according to item 32, wherein the measurement target in which the gas concentration is calculated based on the output of the first gas sensor is the same as the measurement target in which the gas concentration is calculated based on the output of the second gas sensor.

### Item 34

The gas sensor calibration method according to item 32 or 33, wherein

the second gas sensor is provided in a second gas sensor apparatus, and

the second gas sensor apparatus is arranged in an interior space in the measurement target in which the gas concentration is calculated based on the output of the second gas sensor.

### Item 35

The gas sensor calibration method according to item 34, wherein

the first gas sensor is provided in a first gas sensor apparatus,

the transmitting is further transmitting, by the transmission unit, reliability information indicating a calibration reliability of the first gas sensor apparatus,

the receiving is further receiving, by the reception unit, the reliability information transmitted in the transmitting, and

the calibrating is calibrating, by the calibration unit, based on the first calibration information received in the receiving, the gas concentration in the measurement target which is calculated based on the output of the second gas sensor according to the calibration reliability of the first gas sensor apparatus.

### Item 36

The gas sensor calibration method according to item 35, wherein when a reliability of self-calibration of the second gas sensor apparatus is equal to or lower than a predetermined second threshold and also the calibration reliability of the first gas sensor apparatus exceeds a first threshold, the calibrating is calibrating, by the calibration unit, based on the first calibration information received in the receiving, the gas concentration in the measurement target which is calculated based on the output of the second gas sensor.

### Item 37

The gas sensor calibration method according to item 35 or 36, wherein the calibrating is comparing, by the calibration unit, the calibration reliability of the first gas sensor apparatus which is received in the receiving with a calibration reliability of the second gas sensor apparatus, and when the calibration reliability of the first gas sensor apparatus is higher than the calibration reliability of the second gas sensor apparatus, calibrating, by the calibration unit, based on the first calibration information received in the receiving, the gas concentration in the measurement target which is calculated based on the output of the second gas sensor.

### Item 38

The gas sensor calibration method according to item 36 or 37, further comprising:

storing, by a storage unit, the reliability information received in the receiving, wherein

the calibrating is comparing, by the calibration unit, the calibration reliability of the first gas sensor apparatus which is received in the receiving with the calibration reliability of the first gas sensor apparatus which is stored in the storing, and when the calibration reliability of the first gas sensor apparatus which is received in the receiving is higher than the calibration reliability stored in the storing, calibrating, by the calibration unit, based on the first calibration information received in the receiving, the gas concentration in the measurement target which is calculated based on the output of the second gas sensor.

### Item 39

The gas sensor calibration method according to any one of items 36 to 38, further comprising:

acquiring, by a position information acquisition unit, information of a position of the second gas sensor apparatus, wherein

the reliability information includes information of a position of the first gas sensor apparatus, and

the calibrating is calibrating, by the calibration unit, based on the received first calibration information, the gas concentration in the measurement target which is calculated based on the output of the second gas sensor according to a distance between the position of the first gas sensor apparatus and the position of the second gas sensor apparatus which is acquired in the acquiring.

### Item 40

The gas sensor calibration method according to item 39, wherein when the distance is less than a predetermined distance, the calibrating is calibrating, by the calibration unit, based on the received first calibration information, the gas concentration in the measurement target which is calculated based on the output of the second gas sensor.

### Item 41

The gas sensor calibration method according to any one of items 36 to 40, wherein

the receiving is receiving, by the reception unit, first calibration information of each of the plurality of first gas sensor apparatuses, and

the calibrating is calibrating, by the calibration unit, based on the first calibration information of each of the plurality of first gas sensor apparatuses which is received in the receiving, the gas concentration in the measurement target which is calculated based on the output of the second gas sensor.

### Item 42

The gas sensor calibration method according to item 41, wherein

the transmitting is further transmitting, by the transmission unit, the reliability information of each of the plurality of first gas sensor apparatuses,

the receiving is further receiving, by the reception unit, each of the reliability information transmitted in the transmitting, and

the calibrating is applying, by the calibration unit, a weighting to the calibration reliability of each of the plurality of first gas sensor apparatuses, and calibrating, by the calibration unit, based on the calibration reliability to which the weighting has been applied, the gas concentration in the measurement target which is calculated based on the output of the second gas sensor.

### Item 43

The gas sensor calibration method according to item 42, wherein the calibrating is calibrating, by the calibration unit, based on a highest calibration reliability among the calibration reliabilities of each of the plurality of first gas sensor apparatuses, the gas concentration in the measurement target which is calculated based on the output of the second gas sensor.

### Item 44

A gas sensor calibration method comprising:

transmitting, by a transmission unit, first calibration information to a second gas sensor apparatus, wherein

the first calibration information is calibration information for calibrating a gas concentration in a measurement target which is calculated based on an output of a first gas sensor.

### Item 45

The gas sensor calibration method according to item 44, wherein

the second gas sensor apparatus includes a second gas sensor, and

the measurement target in which the gas concentration is calculated based on the output of the first gas sensor is the same as the measurement target in which a gas concentration is calculated based on an output of the second gas sensor.

### Item 46

The gas sensor calibration method according to item 44 or 45, wherein

the first gas sensor is provided in a first gas sensor apparatus, and

the transmitting is further transmitting, by the transmission unit, reliability information indicating a calibration reliability of the first gas sensor apparatus to the second gas sensor apparatus.

### Item 47

A gas sensor calibration method comprising:

receiving, by a reception unit, first calibration information of a first gas sensor apparatus; and

calibrating, by a calibration unit, based on the first calibration information received by the reception unit, a gas concentration in a measurement target which is calculated based on an output of a second gas sensor.

### Item 48

The gas sensor calibration method according to item 47, wherein

the first gas sensor apparatus includes a first gas sensor, and

a measurement target in which a gas concentration is calculated based on an output of the first gas sensor is the same as the measurement target in which the gas concentration is calculated based on the output of the second gas sensor.

### Item 49

The gas sensor calibration method according to item 47 or 48, wherein the second gas sensor is arranged in an interior space in the measurement target in which the gas concentration is calculated based on the output of the second gas sensor.

### Item 50

The gas sensor calibration method according to any one of items 47 to 49, wherein

the receiving is further receiving, by the reception unit, reliability information indicating a calibration reliability of the first gas sensor apparatus, and

the calibrating is calibrating, by the calibration unit, based on the first calibration information received in the receiving, the gas concentration in the measurement target which is calculated based on the output of the second gas sensor according to the calibration reliability of the first gas sensor apparatus.

### Item 51

The gas sensor calibration method according to item 50, wherein

the second gas sensor is provided in a second gas sensor apparatus, and

when a reliability of self-calibration of the second gas sensor apparatus is equal to or lower than a predetermined second threshold and also the calibration reliability of the first gas sensor apparatus exceeds a first threshold, the calibrating is calibrating, by the calibration unit, based on the first calibration information received in the receiving, the gas concentration in the measurement target which is calculated based on the output of the second gas sensor.

### Item 52

The gas sensor calibration method according to item 51, wherein the calibrating is comparing, by the calibration unit, the calibration reliability of the first gas sensor apparatus which is received in the receiving with a calibration reliability of the second gas sensor apparatus, and when the calibration reliability of the first gas sensor apparatus is higher than the calibration reliability of the second gas sensor apparatus, calibrating, by the calibration unit, based on the first calibration information received in the receiving, the gas concentration in the measurement target which is calculated based on the output of the second gas sensor.

### Item 53

The gas sensor calibration method according to item 51 or 52, further comprising:

storing, by a storage unit, the reliability information received in the receiving, wherein

the calibrating is comparing, by the calibration unit, the calibration reliability of the first gas sensor apparatus which is received in the receiving with the calibration reliability of the first gas sensor apparatus which is stored in the storing, and when the calibration reliability of the first gas sensor apparatus which is received in the receiving is higher than the calibration reliability stored in the storing, calibrating, by the calibration unit, based on the first calibration information received in the receiving, the gas concentration in the measurement target which is calculated based on the output of the second gas sensor.

### Item 54

The gas sensor calibration method according to any one of items 51 to 53, further comprising:

acquiring, by a position information acquisition unit, information of a position of the second gas sensor apparatus, wherein

the reliability information includes information of a position of the first gas sensor apparatus, and

the calibrating is calibrating, by the calibration unit, based on the first calibration information received in the receiving, the gas concentration in the measurement target which is calculated based on the output of the second gas sensor according to a distance between the position of the first gas sensor apparatus and the position of the second gas sensor apparatus which is acquired in the acquiring.

### Item 55

The gas sensor calibration method according to item 54, wherein when the distance is less than a predetermined distance, the calibrating is calibrating, by the calibration unit, the gas concentration in the measurement target which is calculated based on the output of the second gas sensor.

### Item 56

The gas sensor calibration method according to any one of items 50 to 55, wherein

the receiving is receiving, by the reception unit, the first calibration information of each of the plurality of first gas sensor apparatuses, and

the calibrating is calibrating, by the calibration unit, based on the first calibration information of each of the plurality of first gas sensor apparatuses which is received in the receiving, the gas concentration in the measurement target which is calculated based on the output of the second gas sensor.

### Item 57

The gas sensor calibration method according to item 56, wherein

the receiving is further receiving, by the reception unit, reliability information of each of the plurality of first gas sensor apparatuses, and

the calibrating is applying, by the calibration unit, a weighting to the calibration reliability of each of the plurality of first gas sensor apparatuses, and calibrating, by the calibration unit, based on the calibration reliability to which the weighting has been applied, the gas concentration in the measurement target which is calculated based on the output of the second gas sensor.

### Item 58

The gas sensor calibration method according to item 53, wherein the calibrating is calibrating, by the calibration unit, based on a highest calibration reliability among the calibration reliabilities of each of the plurality of first gas sensor apparatuses, the gas concentration in the measurement target which is calculated based on the output of the second gas sensor.

### Item 59

A gas sensor calibration program for causing a computer to execute the gas sensor calibration method according to any one of items 32 to 43.

### Item 60

A gas sensor calibration program for causing a computer to execute the gas sensor calibration method according to any one of items 44 to 46.

### Item 61

A gas sensor calibration program for causing a computer to execute the gas sensor calibration method according to any one of items 47 to 58.

### EXPLANATION OF REFERENCES

10: reception unit, 11: first gas sensor, 12: display unit, 13: transmission unit, 14: calibration unit, 15: control unit, 16: position information acquisition unit, 20: reception unit, 21: second gas sensor, 22: display unit, 23: transmission unit, 24: calibration unit, 25: control unit, 26: position information acquisition unit, 30: reception unit, 31: third gas sensor, 32: display unit, 33: transmission unit, 34: calibration unit, 35: control unit, 90: living body, 100: first gas sensor apparatus, 110: computation unit, 112: storage unit, 114: AD conversion unit, 120: computation unit, 122: storage unit, 124: AD conversion unit, 150: gas sensor apparatus, 200: second gas sensor apparatus, 250: gas sensor apparatus, 300: third gas sensor apparatus, 400: gas sensor system, 501: measurement target, 502: measurement target, 503: gas, 504: gas, 505: measurement target, 508: interior space, 600: gas sensor, 2200: computer, 2201: DVD-ROM, 2210: host controller, 2212: CPU, 2214: RAM, 2216: graphics controller, 2218: display device, 2220: input and output controller, 2222: communication interface, 2224: hard disk drive, 2226: DVD-ROM drive, 2230: ROM, 2240: input and output chip, 2242: keyboard.

What is claimed is:

1. A gas sensor system comprising:
a first gas sensor apparatus including a transmission unit configured to transmit first calibration information for calibrating a gas concentration in a measurement target which is calculated based on an output of a first gas sensor, wherein the transmission unit is configured to transmit the first calibration information and reliability information indicating a calibration reliability of the first gas sensor apparatus; and
a second gas sensor apparatus including a reception unit configured to receive the first calibration information and the reliability information which are transmitted by the transmission unit, and a calibration unit configured to calibrate, based on the first calibration information received by the reception unit, a gas concentration in a measurement target which is calculated based on an output of a second gas sensor, wherein
when the calibration reliability of the first gas sensor apparatus which is received by the reception unit is higher than a reference calibration reliability, the calibration unit is configured to calibrate, based on the received first calibration information, the gas concentration in the measurement target which is calculated based on the output of the second gas sensor.

2. The gas sensor system according to claim 1, wherein
the second gas sensor apparatus further includes a storage unit configured to store reference reliability information indicating the reference calibration reliability, and
when the calibration reliability of the first gas sensor apparatus which is received by the reception unit is higher than the reference calibration reliability, the storage unit is configured to update the reference calibration reliability based on the calibration reliability of the first gas sensor apparatus which is received by the reception unit.

3. The gas sensor system according to claim 1, wherein the measurement target in which the gas concentration is calculated based on the output of the first gas sensor is the same as the measurement target in which the gas concentration is calculated based on the output of the second gas sensor.

4. The gas sensor system according to claim 2, wherein the measurement target in which the gas concentration is calculated based on the output of the first gas sensor is the same as the measurement target in which the gas concentration is calculated based on the output of the second gas sensor.

5. The gas sensor system according to claim 1, wherein
the first gas sensor apparatus includes the first gas sensor, and
the first gas sensor apparatus is a mobile terminal.

6. The gas sensor system according to claim 4, wherein the second gas sensor apparatus is arranged in an interior space in the measurement target in which the gas concentration is calculated based on the output of the second gas sensor.

7. The gas sensor system according to claim 1, wherein
the second gas sensor apparatus includes the second gas sensor, and
the second gas sensor apparatus is a mobile terminal.

8. The gas sensor system according to claim 1, wherein when a reliability of self-calibration of the second gas sensor apparatus is equal to or lower than a predetermined second threshold and also the calibration reliability of the first gas sensor apparatus exceeds a first threshold, the calibration unit is configured to calibrate, based on the received first calibration information, the gas concentration in the measurement target which is calculated based on the output of the second gas sensor.

9. The gas sensor system according to claim 1, wherein
the second gas sensor apparatus further includes a position information acquisition unit configured to acquire information of a position of the second gas sensor apparatus,
the reliability information includes information of a position of the first gas sensor apparatus, and
the calibration unit is configured to calibrate, based on the received first calibration information, the gas concentration in the measurement target which is calculated based on the output of the second gas sensor according to a distance between the position of the first gas sensor apparatus and the position of the second gas sensor apparatus which is acquired by the position information acquisition unit.

10. The gas sensor system according to claim 1, comprising:
a plurality of the first gas sensor apparatuses, wherein
the reception unit is configured to receive first calibration information of each of the plurality of first gas sensor apparatuses and the reliability information, and
the calibration unit is configured to apply a weighting to the calibration reliability of each of the plurality of first gas sensor apparatuses, and to calibrate, based on the calibration reliability to which the weighting has been applied, the gas concentration in the measurement target which is calculated based on the output of the second gas sensor.

11. The gas sensor system according to claim 10, wherein the calibration unit is configured to calibrate, based on a highest calibration reliability among the calibration reliabilities of each of the plurality of first gas sensor apparatuses, the gas concentration in the measurement target which is calculated based on the output of the second gas sensor.

12. A gas sensor calibration method comprising:
transmitting, by a transmission unit, first calibration information and reliability information, wherein the first calibration information is calibration information for calibrating a gas concentration in a measurement target which is calculated based on an output of a first gas sensor, and the reliability information is reliability information indicating a calibration reliability of a first gas sensor apparatus;
receiving, by a reception unit, the first calibration information and the reliability information which are transmitted in the transmitting; and
calibrating, by a calibration unit, based on the first calibration information received in the receiving, a gas concentration in a measurement target which is calculated based on an output of a second gas sensor, wherein
when the calibration reliability of the first gas sensor apparatus which is received in the receiving is higher than a reference calibration reliability, the calibrating is calibrating, by the calibration unit, based on the received first calibration information, the gas concentration in the measurement target which is calculated based on the output of the second gas sensor.

13. The gas sensor calibration method according to claim 12, further comprising:
storing, by a storage unit, the reliability information received in the receiving, wherein
when the calibration reliability of the first gas sensor apparatus which is received in the receiving is higher than the reference calibration reliability, the storing includes updating, by the storage unit, the reference calibration reliability based on the calibration reliability of the first gas sensor apparatus which is received in the receiving.

14. The gas sensor calibration method according to claim 12, wherein the measurement target in which the gas concentration is calculated based on the output of the first gas sensor is the same as the measurement target in which the gas concentration is calculated based on the output of the second gas sensor.

15. The gas sensor calibration method according to claim 12, wherein
the second gas sensor is provided in a second gas sensor apparatus, and
the second gas sensor apparatus is arranged in an interior space in the measurement target in which the gas concentration is calculated based on the output of the second gas sensor.

16. The gas sensor calibration method according to claim 12, wherein
the second gas sensor is provided in second gas sensor apparatus, and
when a reliability of self-calibration of the second gas sensor apparatus is equal to or lower than a predetermined second threshold and also the calibration reliability of the first gas sensor apparatus exceeds a first threshold, the calibrating is calibrating, by the calibration unit, based on the first calibration information received in the receiving, the gas concentration in the measurement target which is calculated based on the output of the second gas sensor.

17. The gas sensor calibration method according to claim 12, wherein
the second gas sensor is provided in the second gas sensor apparatus,
the gas sensor calibration method further comprises acquiring, by a position information acquisition unit, information of a position of the second gas sensor apparatus,
the reliability information includes information of a position of the first gas sensor apparatus, and
the calibrating is calibrating, by the calibration unit, based on the received first calibration information, the gas concentration in the measurement target which is calculated based on the output of the second gas sensor according to a distance between the position of the first gas sensor apparatus and the position of the second gas sensor apparatus which is acquired in the acquiring.

18. The gas sensor calibration method according to claim 12, wherein
the receiving is receiving, by the reception unit, first calibration information of each of a plurality of the first gas sensor apparatuses and the reliability information, and
the calibrating is applying, by the calibration unit, a weighting to the calibration reliability of each of the plurality of first gas sensor apparatuses, and calibrating, by the calibration unit, based on the calibration reliability to which the weighting has been applied, the gas concentration in the measurement target which is calculated based on the output of the second gas sensor.

19. The gas sensor calibration method according to claim 18, wherein the calibrating is calibrating, by the calibration unit, based on a highest calibration reliability among the calibration reliabilities of each of the plurality of first gas sensor apparatuses, the gas concentration in the measurement target which is calculated based on the output of the second gas sensor.

20. A computer-readable medium having recorded thereon a program that, when executed by a computer, causes the computer to perform operations comprising:
transmitting first calibration information and reliability information, wherein the first calibration information is calibration information for calibrating a gas concentration in a measurement target which is calculated based on an output of a first gas sensor, and the reliability information is reliability information indicating a calibration reliability of a first gas sensor apparatus;
receiving the first calibration information and the reliability information which are transmitted in the transmitting; and
calibrating, based on the first calibration information received in the receiving, a gas concentration in a measurement target which is calculated based on an output of a second gas sensor, wherein when the calibration reliability of the first gas sensor apparatus which is received in the receiving is higher than a reference calibration reliability, the gas concentration in the measurement target which is calculated based on the output of the second gas sensor is calibrated based on the received first calibration information.
